SEQUENCE LISTING (2 sequences)

SEQ ID NO: 1 - DNA, 19 bp, Artificial Sequence
  5′ to 3′ Passenger polynucleotide
gagacacaugggugcuaua

SEQ ID NO: 2 - DNA, 19 bp, Artificial Sequence
  3′ to 5′ Guide polynucleotide
cucuguguacccacgauau

(19) United States
(12) Patent Application Publication
(10) Pub. No.: US 2024/0077505 A1
(43) Pub. Date: Mar. 7, 2024

(54) LIGAND BINDING ASSAY USING MODIFIED RETINOL BINDING PROTEIN

(71) Applicants: Bristol-Myers Squibb Company, Princeton, NJ (US); Meso Scale Diagnostics, Rockville, MD (US)

(72) Inventors: Ya FU, Princeton, NJ (US); Christopher G. LEVINS, Flemington, NJ (US); Linda J. OBENAUER-KUTNER, Lincroft, NJ (US); Uma KAVITA, Plainsboro, NJ (US); Sharadvi THATI, North Brunswick, NJ (US)

(73) Assignees: Bristol-Myers Squibb Company, Princeton, NJ (US); Meso Scale Diagnostics, Rockville, MD (US)

(21) Appl. No.: 18/258,542

(22) PCT Filed: Dec. 17, 2021
(86) PCT No.: PCT/US2021/064156

§ 371 Date: Jun. 20, 2023

Related U.S. Application Data
(60) Provisional application No. 63/127,877, filed on Dec. 18, 2020.

(51) Int. Cl. G01N 33/92; G01N 33/566

(52) U.S. Cl. CPC G01N 33/92; G01N 33/566

(57) ABSTRACT

The disclosure provides a method of measuring the relative RBP-binding affinity of a liposome in a sample. In some aspects, the method comprises contacting the sample with a modified RBP associated with an immobilized surface, wherein an exterior surface of the liposome comprises a retinoid or a fat-soluble vitamin, and wherein the modified RBP binds the retinoid or the fat-soluble vitamin.

This application claims priority to and benefit of U.S. Provisional Application No. 63/127,877, filed on Dec. 18, 2020, which is hereby incorporated by reference herein in its entirety.

### REFERENCE TO SEQUENCE LISTING SUBMITTED ELECTRONICALLY VIA EFS-WEB

The content of the electronically submitted sequence listing (Name: 3338_207PC01_Seqlisting_ST25.txt; Size: 634 Bytes; and Date of Creation: Dec. 14, 2021) is herein incorporated by reference in its entirety.

### FIELD OF THE DISCLOSURE

The present disclosure provides methods for assaying the bioperformance of vitamin A-conjugated LNP targeting moieties.

### BACKGROUND OF THE DISCLOSURE

Therapeutic RNAs are a valuable tool in targeted treatment of a variety of diseases and conditions. However, RNA molecules are notoriously unstable, and protecting therapeutic RNAs while targeting the RNAs to a particular region is crucial to their efficacy. Encapsulating the RNA in a lipid nanoparticle (LNP) comprising a targeting moiety on its surface can achieve both goals. Vitamin A-conjugated targeting agents (such as DiVA-PEG-DiVA, or DPD) have emerged as a means of targeting lipid nanoparticles to activated hepatic stellate cells (HSCs). The DPD component enhances potency and effectiveness of siRNA delivery by binding to the retinol-binding protein (RBP) in plasma. The RBP-bound LNP is then internalized by HSCs via the RBP receptor, and target gene expression in the HSCs can then be inhibited via the RNAi pathway.

The DPD component of the LNP is sensitive to UV and white light (indoor light), which negatively impact the targeting ability of the DPD. Isomerization and chain oxidation reaction are the main degradation pathways induced by UV/white light exposure. Contemporary methods for monitoring the target bioperformance of DPD comprising drug products, such as enzyme linked immunosorbent assay (ELISA), surface plasmon resonance (SPR) by Biacore, and Western blot analysis, have been unsuccessful, at least in part due to non-specific signals intrinsic to the LNP. As such there remains a need for improved methods of measuring the functionality of Vitamin A-conjugated targeting agents as part of an LNP drug product.

### SUMMARY OF THE DISCLOSURE

Some aspects of the present disclosure are directed to a method for detecting a liposome in a sample, comprising contacting the sample with a modified retinol binding protein (RBP) associated with an immobilized surface; wherein an exterior surface of the liposome comprises a retinoid or a fat-soluble vitamin; and wherein the modified RBP binds the retinoid or the fat-soluble vitamin.

Some aspects of the present disclosure are directed to a method of measuring the relative RBP-binding affinity of a liposome in a sample, comprising contacting the sample with a modified RBP associated with an immobilized surface; wherein an exterior surface of the liposome comprises a retinoid or a fat-soluble vitamin; and wherein the modified RBP binds the retinoid or the fat-soluble vitamin.

In some aspects, the retinoid or the fat-soluble vitamin comprises a vitamin A or a DiVA-PEG-DiVA (DPD) construct.

In some aspects, the modified RBP comprises a heterologous moiety, and wherein the immobilized surface comprises a binding element that is capable of binding the heterologous moiety. In some aspects, the heterologous moiety comprises biotin. In some aspects, the binding element comprises avidin, streptavidin, NeutrAvidin, CaptAvidin, or any combination thereof. In some aspects, the modified RBP comprises a biotin, and wherein the immobilized surface comprises avidin, streptavidin, NeutrAvidin, or any combination thereof.

In some aspects, the immobilized surface is a plate. In some aspects, the plate comprises one or more electrodes associated with the bottom surface of the plate. In some aspects, the immobilized surface is a magnetic bead or a chip.

In some aspects, the liposome is a lipid nanoparticle (LNP). In some aspects, the liposome does not comprise a biotin.

In some aspects, the method further comprises contacting the liposome with an antibody that binds an antigen present on the liposome. In some aspects, the antibody specifically binds PEG (“an anti-PEG antibody”). In some aspects, the anti-PEG antibody specifically binds a methoxy group of PEG. In some aspects, the anti-PEG antibody is conjugated to a detectable tag.

In some aspects, the liposome comprises a biologically active molecule. In some aspects, the biologically active molecule is a chemical compound, a nucleic acid, a peptide, an amino acid, or any combination thereof. In some aspects, the biologically active molecule is DNA, RNA, or any combination thereof. In some aspects, the biologically active molecule is an antisense oligonucleotide, siRNA, shRNA, miRNA, mRNA, a DNA plasmid, Cas9 nuclease, a TALEN nuclease, a zinc finger nuclease, or any combination thereof. In some aspects, the biologically active molecule inhibits heat shock protein 47 (HSP47). In some aspects, the biologically active molecule is an siRNA. In some aspects, the siRNA comprises the nucleotide sequence set forth in SEQ ID NO: 1.

In some aspects, the method comprises treating the immobilized surface with a blocker solution prior to the contacting with the sample. In some aspects, the blocker solution comprises a polysorbate. In some aspects, the blocker solution comprises about 0.001% to about 0.05% TWEEN-20. In some aspects, the blocker solution comprises at least about 0.01% TWEEN-20. In some aspects, the blocker solution comprises about 0.05% TWEEN-20.

In some aspects, the method further comprises washing the immobilized surface with a wash buffer prior to the contacting with the sample. In some aspects, the method comprises washing the immobilized surface at least one time, at least two times, at least three times, at least four times, or at least five times with the wash buffer. In some aspects, the method comprises washing the immobilized surface three times with the wash buffer. In some aspects, the wash buffer comprises phosphate buffered saline (PBS) and TWEEN-20. In some aspects, the wash buffer comprises about 0.001% to about 0.05% TWEEN-20. In some aspects, the wash solution comprises at least about 0.01% TWEEN-20. In some aspects, the wash solution comprises about 0.05% TWEEN-20.

In some aspects, the method further comprises washing the immobilized surface with a wash buffer after the contacting with the sample. In some aspects, the method further comprises washing the immobilized surface at least one time, at least two times, at least three times, at least four times, or at least five times with the wash buffer. In some aspects, the method comprises washing the immobilized surface three times with the wash buffer. In some aspects, the wash buffer comprises PBS and TWEEN-20. In some aspects, the wash buffer comprises about 0.001% to about 0.05% TWEEN-20. In some aspects, the wash buffer comprises at least about 0.01% TWEEN-20. In some aspects, the wash buffer comprises about 0.05% TWEEN-20.

In some aspects, the method further comprises contacting the immobilized surface with a detection antibody solution. In some aspects, the detection antibody solution comprises an antibody that specifically binds an antigen present on a lipid-based particle. In some aspects, the antibody specifically binds PEG (“an anti-PEG antibody”). In some aspects, the anti-PEG antibody specifically binds a methoxy group of PEG. In some aspects, the antibody is conjugated to a detectable tag.

In some aspects, the method further comprises washing the immobilized surface with a wash buffer. In some aspects, the method comprises washing the immobilized surface at least one time, at least two times, at least three times, at least four times, or at least five times with the wash buffer. In some aspects, the method comprises washing the immobilized surface three times with the wash buffer. In some aspects, the wash buffer comprises PBS and TWEEN-20. In some aspects, the wash buffer comprises about 0.001% to about 0.05% TWEEN-20. In some aspects, the wash buffer comprises at least about 0.01% TWEEN-20. In some aspects, the wash buffer comprises about 0.05% TWEEN-20. In some aspects, the method further comprises detecting the antibody.

In some aspects, the method further comprises administering the liposome to a subject in need thereof.

In some aspects, the liposome is capable of preventing or treating a disease or condition in a subject. In some aspects, the disease or condition affects the liver of the subject. In some aspects, the disease or condition is selected from hepatic cirrhosis, liver fibrosis, Nonalcoholic Steatohepatitis (NASH), alcoholic steatohepatitis, primary sclerosing cholangitis, primary biliary cirrhosis, and any combination thereof. In some aspects, the disease or condition comprises an organ fibrosis. In some aspects, the organ fibrosis is selected from liver fibrosis, lung fibrosis, a glial scar, arterial stiffness, arthrofibrosis, Crohn's disease, Dupuytren's contracture, keloid formation, mediastinal fibrosis, myelofibrosis, peyronie's disease, nephrogenic systemic fibrosis, progressive massive fibrosis, retroperitoneal fibrosis, scleroderma/systemic sclerosis, adhesive capsulitis, and any combination thereof.

Some aspects of the present disclosure are directed to a kit for measuring the relative RBP-binding affinity of a liposome in a sample according to any method disclosed herein. Some aspects of the present disclosure are directed to a kit for assaying the relative RBP-targeting bioperformance of a liposome in a sample according to any method disclosed herein.

### BRIEF DESCRIPTION OF THE DRAWINGS

FIG. 1 is a schematic drawing of a single-plex sandwich immunoassay using carbon electrode microtiter plates (streptavidin (SA) coated) and ECL detection measured on an MSD Sector Imager plate reader (Meso Scale Diagnostics, LLC.). SA=streptavidin. PEG=polyethylene glycol.

FIGS. 2A-2B are graphical representations of LNP titration curves using increasing concentrations of DPD-naked LNPs (DPD(−)) or LNPs with surface DPD (DPD(+)) using a wash buffer comprising 0.01% Tween-20 in PBS (PBST; FIG. 2A) or 0.05% PB ST (FIG. 2B).

FIGS. 3A-3B are graphical representations illustrating the integrity of LNPs after exposure to increasing concentrations of Tween-20 (PBST; FIG. 3A) and over time (FIG. 3B). FIG. 3A is a bar graph illustrating the percent of siRNA remaining after incubation of LNP samples in ST buffer with different amounts of Tween20 (0.0%, 0.01%, 0.05%, 0.1%, 1% and 5%) for 30 minutes, than analyzed by RiboGreen assay. FIG. 3B is a line graph illustrating the percent of siRNA remaining in LNP incubated in 0.01% PB ST buffer for up to 120 minutes.

FIGS. 4A-4B are graphical representations of RBP binding curves for two samples. The relative percent RBP-binding was calculated by comparing the normalized EC50 value to the reference standard (positive LNP).

FIGS. 5A-5B are line graphs illustrating the precision and accuracy of the RBP binding of several samples from two clinical lots (FIG. 5A) and a negative control (FIG. 5B) across 9 runs.

FIG. 6 is a graphical representation of linearity of RBP binding relative to LNP concentration using seven samples with starting concentration of 87.5, 75, 62.5, 50, 37.5, 25 and 12.5 ug/mL prepared from reference standard for three separate runs. The slope of the regression line between the relative concentrations and their measured relative % RBP-binding represents linearity.

FIG. 7 is a graphical representation of the relative RBP-binding of HSP47 LNP having increased concentrations of surface DPD across two runs.

### DETAILED DESCRIPTION OF THE DISCLOSURE

The present disclosure is directed to methods of measuring the relative RBP-binding affinity of a liposome in a sample, comprising contacting the sample with a modified RBP associated with an immobilized surface. In some aspects, an exterior surface of the liposome comprises a retinoid or a fat-soluble vitamin, and the modified RBP binds the retinoid or the fat-soluble vitamin. In certain aspects, the methods disclosed herein are used to monitor the concentration and/or stability of a liposome comprising a surface exposed retinoid or fat-soluble vitamin. In some aspects, the retinoid or fat-soluble vitamin comprises a vitamin A, an analog thereof, or a derivative thereof.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

A “liposome,” as used herein, refers to a spherical vesicle, comprising one or more phospholipid layer. Liposomes can range in size from about 0.025 μm to about 2.5 μm. In some aspects, the liposome comprises a phospholipid bilayer. In other aspects, the liposome comprises a phospholipid monolayer. On the basis of their size and number of bilayers, liposomes can be classified into one of two categories: (1) multilamellar vesicles (MLV), having more than one lipid bilayer, and (2) unilamellar vesicles, having only one lipid bylayer. Unilamellar vesicles can also be classified into two categories: (1) large unilamellar vesicles (LUV) and (2) small unilamellar vesicles (SUV) (see Akbarzadeh et al., nanoscale Res. Lett. 8 (1):102 (2013), which is incorporated by reference herein in its entirety). In unilamellar liposomes, the vesicle has a single phospholipid bilayer sphere enclosing the aqueous solution. In multilamellar liposomes, vesicles have an onion structure. Liposomes disclosed herein can be naturally occurring, e.g., isolated from a cell, or artificial. Non-limiting examples of liposomes are disclosed in Puri et al, Crit. Rev. Ther. Drug Carrier Syst. 26 (6):523-80(2009), which is incorporated by reference herein in its entirety.

In certain aspects, the liposome is a lipid nanoparticle. As used herein, the terms “lipid nanoparticle,” “lipid-based nanoparticle,” and “LNP,” are used interchangeably and refer to a type of liposome-like vesicle, which is composed primarily of cationic lipids, neutral phosphatidylcholine lipids, sterols (including cholesterol), and PEGylated phospholipids.

“Binding affinity,” e.g., “RBP-binding affinitiy,” as used herein, refers to the ability and/or strength of one moiety, e.g., a retinoid, to interact with a second moiety, e.g., an RBP.

“Retinol binding protein” or “RBP,” as used herein, refers to a family of polypeptides capable of binding retinol. RBP is hepatically synthesized and responsible for transporting vitamin A from the liver to other tissues. RBP is capable of binding vitamin A, retinoids, derivatives, and conjugates thereof. Any RBP known in the art, or a retinol-binding portion thereof can be used in the methods disclosed herein. In some aspects, the RBP is a human RBP, or a retinol-binding portion thereof. In humans, there are at least 6 RBPs, including RBP1 (UniProtKB—P09455), RBP2 (UniProtKB—P50120), RBP3 (UniProtKB—P10745), RBP4 (UniProtKB—P02753), RBP5 (UniProtKB—P82980), and RBP7 (UniProtKB—Q96R05). In certain aspects, the RBP is derived from a human RBP. In some aspects, the RBP is a non-human primate RBP. In some aspects, the RBP is a mouse or rat RBP, or a retinol-binding portion thereof. In some aspects, the RBP is derived from a mouse or rat RBP.

A “fat-soluble vitamin,” as used herein, refers to a vitamin selected from vitamins A, D, E, K, and derivatives thereof. In certain aspects, the fat-soluble vitamin comprises vitamin A or a derivative thereof. Non-limiting examples of vitamin A derivatives and analogs sutiable for the methods disclosed herein are disclosed in U.S. Publication No. 2013/0171240 A1, which is incorporated by reference herein in its entirety. In certain aspects, the fat-soluble vitamin comprises a DiVA motif. In certain aspects, the fat-soluble vitamin comprises a DiVa-PEG-DiVa (N1,N19-bis((S,23E,25E,27E,29E)-16-((2E,4E,6E,8E)-3,7-dimethyl-9-(2,6,6-trimethylcyclo-hex-1-en-1-yl)nona-2,4,6,8-tetraenamido)-24,28-dimethyl-15,22-dioxo-30-(2,6,6-trimethylcyclohexl-en-1-yl)-4,7,10-trioxa-14,21-diazatriaconta-23,25,27,29-tetraen-1-yl)-4,7,10,13,16-pentaoxanonadecane-1,19-diamide).

A “biologically active molecule,” as used herein, refers to any substance which can affect any physical or biochemical properties of a biological system, pathway, molecule, or interaction relating to a living organism. In particular, as used herein, biologically active molecules include, but are not limited to, any substance intended for diagnosis, cure, mitigation, treatment, or prevention of disease or conditions, e.g., diseases or conditions associated with fibrosis, in humans or other animals, or to otherwise enhance physical or mental well-being of humans or animals. Biologically active molecules can comprise a polypeptide (e.g., a protein (e.g., an enzyme, a growth factor, a cytokine, a chemokine, a ligand, a receptor, a hormone, an antibody, an antigen, a fragment thereof, or any combination thereof), a nucleotide (e.g., a DNA (e.g., a ssDNA, a dsDNA, a plasmid, a fragment thereof, or any combination thereof) an RNA (e.g., a mRNA, a miRNA, an siRNA, a dsRNA, a fragment thereof, or any combination thereof), an antisense oligomer, a fragment thereof, or any combination thereof), a Cas9 nuclease, a TALEN nuclease, a zinc finger nuclease, a small molecule, or any combination thereof.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as VL) and a light chain constant region. The light chain constant region comprises one constant domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

The term “antigen-binding portion” or “antigen-binding fragment” of an antibody, as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment (fragment from papain cleavage) or a similar monovalent fragment consisting of the V_{L}, V_{H}, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR) and (vii) a combination of two or more isolated CDRs which can optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range, or integer range is to be understood to include the value of any integer within the recited range and, when appropriate, fractions thereof (such as one tenth and one hundredth of an integer), unless otherwise indicated.

As used herein, the terms “ug,” “uM,” and “uL” are used interchangeably with “μg,” “μM,” and “μL” respectively.

Various aspects of the disclosure are described in further detail in the following subsections.

### II. Methods of the Disclosure

Certain aspects of the present disclosure are directed to methods of measuring the relative retinol binding protein (RBP)-binding affinity of a liposome, wherein an exterior surface of the liposome comprises a retinoid or a fat-soluble vitamin. Some aspects of the present disclosure are directed to methods of detecting a liposome in a sample, wherein an exterior surface of the liposome comprises a retinoid or a fat-soluble vitamin. In some aspects, the method comprises contacting the sample with a modified RBP associated with an immobilized surface, wherein the modified RBP binds the retinoid or the fat-soluble vitamin. In some aspects, the interaction between the liposome, e.g., LNP, and the RBP is measured using an antibody or antigen-binding fragment thereof that specifically binds an antigen on the liposome, e.g., LNP.

### II.A. Blocker Solution

In certain aspects, the immobilized surface is treated prior to contacting the sample with the immobilized surface. In some aspects, the immobilized surface is treated with a blocker solution.

In some aspects, the blocker solution does not comprise a polysorbate. In other aspects, the blocker solution comprises a polysorbate. In certain aspects, the blocker solution comprises polysorbate 20 (e.g., TWEEN-20), polysorbate 80 (e.g., TWEEN-80), or both. In some aspects, the blocker solution comprises at least about 0.001% polysorbate 20 (e.g., TWEEN-20). In some aspects, the blocker solution comprises at least about 0.001%, at least about 0.005%, at least about 0.01%, at least about 0.05%, at least about 0.1%, at least about 0.5%, or at least about 1% polysorbate 20 (e.g., TWEEN-20). In some aspects, the blocker solution comprises at least about 0.001% to at least about 1%, at least about 0.001% to at least about 0.5%, at least about 0.001% to at least about 0.1%, at least about 0.001% to at least about 0.05%, at least about 0.001% to at least about 0.01%, at least about 0.01% to at least about 1%, at least about 0.01% to at least about 0.5%, at least about 0.01% to at least about 0.1%, or at least about 0.05% to at least about 1% polysorbate 20 (e.g., TWEEN-20). In some aspects, the blocker solution comprises at least about 0.001% polysorbate 20 (e.g., TWEEN-20). In some aspects, the blocker solution comprises at least about at least about 0.005% polysorbate 20 (e.g., TWEEN-20). In some aspects, the blocker solution comprises at least about at least about 0.01% polysorbate 20 (e.g., TWEEN-20). In some aspects, the blocker solution comprises at least about at least about 0.05% polysorbate 20 (e.g., TWEEN-20). In some aspects, the blocker solution comprises at least about at least about 0.1%, at least about 0.5% polysorbate 20 (e.g., TWEEN-20). In some aspects, the blocker solution comprises at least about or at least about 1% polysorbate 20 (e.g., TWEEN-20).

The blocker solution can be applied to the immobilized surface for any period of time. In some aspects, the blocker solution is applied to the immobilized surface for at least about 30 minutes to at least about 24 hours, from at least about 1 hour to at least about 12 hours, from at least 1 hour to at least about 9 hours, from at least about 1 hour to at least about 6 hours, from at least 1 hour to at least about 5 hours, from at least 1 hour to at least about 4 hours, from at least 1 hour to at least about 3 hours, or from at least 1 hour to at least about 2 hours. In some aspects, the sample is contacted with the immobilized surface for at least about 30 minutes. In some aspects, the blocker solution is applied to the immobilized surface for at least about 45 minutes. In some aspects, the blocker solution is applied to the immobilized surface for at least about 60 minutes. In some aspects, the blocker solution is applied to the immobilized surface for at least about 90 minutes. In some aspects, the blocker solution is applied to the immobilized surface for at least about 120 minutes. In some aspects, the blocker solution is applied to the immobilized surface for at least about 150 minutes. In some aspects, the blocker solution is applied to the immobilized surface for at least about 180 minutes. In some aspects, the blocker solution is applied to the immobilized surface for at least about four hours. In some aspects, the blocker solution is applied to the immobilized surface for at least about five hours. In some aspects, the blocker solution is applied to the immobilized surface for at least about six hours. In some aspects, the blocker solution is applied to the immobilized surface for at least about nine hours. In some aspects, the blocker solution is applied to the immobilized surface for at least about twelve hours. In some aspects, the blocker solution is applied to the immobilized surface for at least about twenty-four hours. In certain aspects, the blocker solution is applied to the immobilized surface overnight.

In some aspects, the immobilized surface is subjected to an agitation while the blocker solution is applied to the immobilized surface. In some aspects, the agitation comprises a rocking, a shaking, a turning, a rotating, a spinning, or any combination thereof. In certain aspects, the immobilized surface is rocked while the blocker solution is applied to the immobilized surface. In some aspects, the immobilized surface is rocked on a plate shaker while the blocker solution is applied to the immobilized surface, wherein the plate shaker is set to a speed of less than about 1000 rpm, less than about 900 rpm, less than about 800 rpm, less than about 700 rpm, less than about 600 rpm, less than about 500 rpm, less than about 450 rpm, less than about 400 rpm, less than about 350 rpm, less than about 300 rpm, less than about 250 rpm, or less than about 200 rpm. In certain aspects, the immobilized surface is rocked on a plate shaker while the blocker solution is applied to the immobilized surface, wherein the plate shaker is set to a speed of about 400 rpm. In certain aspects, the immobilized surface is rocked on a plate shaker while the blocker solution is applied to the immobilized surface, wherein the plate shaker is set to a speed of about 375 rpm. In certain aspects, the immobilized surface is rocked on a plate shaker while the blocker solution is applied to the immobilized surface, wherein the plate shaker is set to a speed of about 350 rpm. In certain aspects, the immobilized surface is rocked on a plate shaker while the blocker solution is applied to the immobilized surface, wherein the plate shaker is set to a speed of about 325 rpm. In certain aspects, the immobilized surface is rocked on a plate shaker while the blocker solution is applied to the immobilized surface, wherein the plate shaker is set to a speed of about 300 rpm.

In some aspects, the blocker solution is applied to the immobilized surface at room temperature. In some aspects, the blocker solution is applied to the immobilized surface at less than room temperature. In some aspects, the blocker solution is applied to the immobilized surface at higher than room temperature. In some aspects, the blocker solution is applied to the immobilized surface at a temperature of at least about 37° C. In some aspects, the blocker solution is applied to the immobilized surface at a temperature of less than about 37° C. In some aspects, the blocker solution is applied to the immobilized surface at a temperature from at least about 4° C. to at least about 20° C. In some aspects, the blocker solution is applied to the immobilized surface at a temperature of at least about 4° C. In some aspects, the blocker solution is applied to the immobilized surface at a temperature of less than about 4° C.

### II.B. Contacting the Sample with the Immobilized Surface

In certain aspect, the sample comprising the liposome is contacted with the immobilized surface after the immobilized surface is treated with the blocking solution. The sample can be contacted with the immobilized surface under any conditions that enhance an interaction between the liposome and the immobilized surface. In some aspects, the sample is contacted with the immobilized surface at room temperature. In some aspects, the sample is contacted with the immobilized surface at less than room temperature. In some aspects, the sample is contacted with the immobilized surface at higher than room temperature. In some aspects, the sample is contacted with the immobilized surface at a temperature of at least about 37° C. In some aspects, the sample is contacted with the immobilized surface at a temperature of less than about 37° C. In some aspects, the sample is contacted with the immobilized surface at a temperature of at least about 4° C.

The sample can be contacted with the immobilized surface for any period of time sufficient to allow for the association between the liposomes and the immobilized surface. In some aspects, the sample is contacted with the immobilized surface from at least about 30 minutes to at least about 24 hours, from at least about 1 hour to at least about 12 hours, from at least 1 hour to at least about 9 hours, from at least about 1 hour to at least about 6 hours, from at least 1 hour to at least about 5 hours, from at least 1 hour to at least about 4 hours, from at least 1 hour to at least about 3 hours, or from at least 1 hour to at least about 2 hours. In some aspects, the sample is contacted with the immobilized surface for at least about 30 minutes. In some aspects, the sample is contacted with the immobilized surface for at least about 45 minutes. In some aspects, the sample is contacted with the immobilized surface for at least about 60 minutes. In some aspects, the sample is contacted with the immobilized surface for at least about 90 minutes. In some aspects, the sample is contacted with the immobilized surface for at least about 120 minutes. In some aspects, the sample is contacted with the immobilized surface for at least about 150 minutes. In some aspects, the sample is contacted with the immobilized surface for at least about 180 minutes. In some aspects, the sample is contacted with the immobilized surface for at least about four hours. In some aspects, the sample is contacted with the immobilized surface for at least about five hours. In some aspects, the sample is contacted with the immobilized surface for at least about six hours. In some aspects, the sample is contacted with the immobilized surface for at least about nine hours. In some aspects, the sample is contacted with the immobilized surface for at least about twelve hours. In some aspects, the sample is contacted with the immobilized surface for at least about twenty-four hours. In certain aspects, the sample is contacted with the immobilized surface overnight.

In some aspects, the immobilized surface is subjected to an agitation while the sample is contacted with the immobilized surface. In some aspects, the agitation comprises a rocking, a shaking, a turning, a rotating, a spinning, or any combination thereof. In certain aspects, the immobilized surface is rocked while the sample is contacted with the immobilized surface. In some aspects, the agitation comprises a rocking, a shaking, a turning, a rotating, a spinning, or any combination thereof. In certain aspects, the immobilized surface is rocked while the sample is contacted with the immobilized surface. In some aspects, the immobilized surface is rocked on a plate shaker while the sample is contacted with the immobilized surface, wherein the plate shaker is set to a speed of less than about 1000 rpm, less than about 900 rpm, less than about 800 rpm, less than about 700 rpm, less than about 600 rpm, less than about 500 rpm, less than about 450 rpm, less than about 400 rpm, less than about 350 rpm, less than about 300 rpm, less than about 250 rpm, or less than about 200 rpm. In certain aspects, the plate shaker is set to a speed of about 400 rpm. In certain aspects, the plate shaker is set to a speed of about 375 rpm. In certain aspects, the plate shaker is set to a speed of about 350 rpm. In certain aspects, the plate shaker is set to a speed of about 325 rpm. In certain aspects, the plate shaker is set to a speed of about 300 rpm.

### II.C. Detection Antibody

In some aspects, the methods disclosed herein further comprise detecting the presence of a liposome, e.g., LNP, bound to the immobilized surface. Any means of detection known in the art can be used in the methods disclosed herein. In some aspects, the method comprises contacting the liposome, e.g., LNP, with an antibody or an antigen-binding fragment thereof that binds an antigen on the surface of the liposome, e.g., LNP. In some aspects, the antibody or the antigen-binding fragment thereof binds a PEG present on the surface of the liposome, e.g., LNP. In certain aspects, the antibody or the antigen binding portion thereof specifically binds a methoxy group of the PEG. In certain aspects, the antibody or the antigen binding portion thereof does not bind a polysorbate (e.g., TWEEN 20 and/or TWEEN 80).

In certain aspects, the antibody or the antigen-binding fragment thereof comprises a detectable tag. In some aspects, the antibody or the antigen binding portion thereof comprises a detectable tag selected from the group consisting of a fluorescent marker, a substrate, a radiolabel, and any combination thereof.

In some aspects, the method further comprises contacting the immobilized surface with a detection antibody solution after the sample comprising the liposome, e.g., LNP, has been applied to the immobilized surface. In some aspects, the antibody solution is applied after the immobilized surface has been washed. In some aspects, the antibody solution comprises an antibody or an antigen-binding fragment thereof that specifically binds an antigen on the liposome, e.g., LNP. In some aspects, the antibody is any antibody disclosed herein, e.g., an anti-PEG antibody that specifically binds a methoxy group of PEG.

In some aspects, the immobilized surface is incubated in the antibody solution. In some aspects, the immobilized surface is incubated in the antibody solution for at least about 30 minutes, at least about 60 minutes, at least about 90 minutes, at least about 120 minutes, at least about 2.5 hours, at least about 3 hours, at least about 4 hours, at least about 5 hours, at least about 6 hours, at least about 9 hours, at least about 12 hours, at least about 15 hours, at least about 18 hours, at least about 21 hours, or at least about 24 hours in the antibody solution. In certain aspects, the immobilized surface is incubated in the antibody solution for at least about 60 minutes in the antibody solution. In certain aspects, the immobilized surface is incubated in the antibody solution for at least about 120 minutes in the antibody solution. In certain aspects, the immobilized surface is incubated in the antibody solution for at least about 2 hours in the antibody solution. In certain aspects, the immobilized surface is incubated in the antibody solution for at least about 3 hours in the antibody solution. In certain aspects, the immobilized surface is incubated in the antibody solution for at least about 4 hours in the antibody solution. In certain aspects, the immobilized surface is incubated in the antibody solution for at least about 5 hours in the antibody solution. In certain aspects, the immobilized surface is incubated in the antibody solution for at least about 6 hours in the antibody solution. In certain aspects, the immobilized surface is incubated in the antibody solution for at least about 12 hours in the antibody solution. In certain aspects, the immobilized surface is incubated in the antibody solution overnight.

In certain aspects, the immobilized surface is incubated in the antibody solution at room temperature. In certain aspects, the immobilized surface is incubated in the antibody solution at lower than room temperature. In certain aspects, the immobilized surface is incubated in the antibody solution at a temperature from at least about 4° C. to at least about 37° C. In certain aspects, the immobilized surface is incubated in the antibody solution at a temperature of at least about 4° C. In certain aspects, the immobilized surface is incubated in the antibody solution at a temperature of less than about 37° C. In certain aspects, the immobilized surface is incubated in the antibody solution at a temperature of at least about 37° C.

In some aspects, the immobilized surface is subjected to an agitation while the immobilized surface is incubated in the antibody solution. In some aspects, the agitation comprises a rocking, a shaking, a turning, a rotating, a spinning, or any combination thereof. In certain aspects, the immobilized surface is rocked while the immobilized surface is incubated in the antibody solution. In some aspects, the agitation comprises a rocking, a shaking, a turning, a rotating, a spinning, or any combination thereof. In certain aspects, the immobilized surface is rocked while the immobilized surface is incubated in the antibody solution. In some aspects, the immobilized surface is rocked on a plate shaker while the immobilized surface is incubated in the antibody solution, wherein the plate shaker is set to a speed of less than about 1000 rpm, less than about 900 rpm, less than about 800 rpm, less than about 700 rpm, less than about 600 rpm, less than about 500 rpm, less than about 450 rpm, less than about 400 rpm, less than about 350 rpm, less than about 300 rpm, less than about 250 rpm, or less than about 200 rpm. In certain aspects, the plate shaker is set to a speed of about 400 rpm. In certain aspects, the plate shaker is set to a speed of about 375 rpm. In certain aspects, the plate shaker is set to a speed of about 350 rpm. In certain aspects, the plate shaker is set to a speed of about 325 rpm. In certain aspects, the plate shaker is set to a speed of about 300 rpm.

In some aspects, the method further comprises detecting the antibody, e.g., the anti-PEG antibody. In some aspects, the antibody comprises a detectable tag. In some aspects, the antibody is detected by measuring the presence and or intensity of the detectable tag. In some aspects, the antibody is detected by applying a second antibody that binds a constant region of the antibody.

### II.D. Wash Buffers

In some aspects, the method further comprises washing the immobilized surface with a wash buffer. The immobilized surface can be washed before and/or after any step disclosed herein. In some aspects, the immobilized surface is washed prior to applying the blocking solution. In some aspects, the immobilized surface is washed after to applying the blocking solution. In some aspects, the immobilized surface is washed prior to applying the sample. In some aspects, the immobilized surface is washed after applying the sample. In some aspects, the immobilized surface is washed prior to incubation in the antibody solution. In some aspects, the immobilized surface is washed after incubation in the antibody solution. In some aspects, the immobilized surface is washed after each step. In some aspects, the immobilized surface is washed at least one time, at least two times, at least three times, at least four times, or at least five times with the wash buffer. In certain aspects, the immobilized surface is washed three times with the wash buffer.

In some aspects, the wash buffer comprises phosphate buffered saline (PBS). In some aspects, the wash buffer comprise a polysorbate. In some aspects, the wash buffer comprises PBS and a polysorbate. In some aspects, the wash buffer consists of or consists essentially of PBS and a polysorbate. In some aspects, the wash buffer comprises polysorbate 20 (e.g., TWEEN-20), polysorbate 80 (e.g., TWEEN-80), or both. In some aspects, the wash buffer comprises at least about 0.001% polysorbate 20 (e.g., TWEEN-20). In some aspects, the wash buffer comprises at least about 0.001%, at least about 0.005%, at least about 0.01%, at least about 0.05%, at least about 0.1%, at least about 0.5%, or at least about 1% polysorbate 20 (e.g., TWEEN-20). In some aspects, the wash buffer comprises at least about 0.001% to at least about 1%, at least about 0.001% to at least about 0.5%, at least about 0.001% to at least about 0.1%, at least about 0.001% to at least about 0.05%, at least about 0.001% to at least about 0.01%, at least about 0.01% to at least about 1%, at least about 0.01% to at least about 0.5%, at least about 0.01% to at least about 0.1%, or at least about 0.05% to at least about 1% polysorbate 20 (e.g., TWEEN-20). In some aspects, the wash buffer comprises about 0.001% to about 0.05% polysorbate 20 (e.g., TWEEN-20). In some aspects, the wash buffer comprises at least about 0.01% polysorbate 20 (e.g., TWEEN-20). In some aspects, the wash buffer comprises about 0.05% polysorbate 20 (e.g., TWEEN-20). In some aspects, the wash buffer comprises at least about 0.1% polysorbate 20 (e.g., TWEEN-20). In some aspects, the wash buffer comprises at least about 0.5% polysorbate 20 (e.g., TWEEN-20).

In some aspects, the wash buffer comprises at least about 0.001% polysorbate 80 (e.g., TWEEN-80). In some aspects, the wash buffer comprises at least about 0.001%, at least about 0.005%, at least about 0.01%, at least about 0.05%, at least about 0.1%, at least about 0.5%, or at least about 1% polysorbate 80 (e.g., TWEEN-80). In some aspects, the wash buffer comprises at least about 0.001% to at least about 1%, at least about 0.001% to at least about 0.5%, at least about 0.001% to at least about 0.1%, at least about 0.001% to at least about 0.05%, at least about 0.001% to at least about 0.01%, at least about 0.01% to at least about 1%, at least about 0.01% to at least about 0.5%, at least about 0.01% to at least about 0.1%, or at least about 0.05% to at least about 1% polysorbate 80 (e.g., TWEEN-80). In some aspects, the wash buffer comprises about 0.001% to about 0.05% polysorbate 80 (e.g., TWEEN-80). In some aspects, the wash buffer comprises at least about 0.01% polysorbate 80 (e.g., TWEEN-80). In some aspects, the wash buffer comprises about 0.05% polysorbate 80 (e.g., TWEEN-80). In some aspects, the wash buffer comprises at least about 0.1% polysorbate 80 (e.g., TWEEN-80). In some aspects, the wash buffer comprises at least about 0.5% polysorbate 80 (e.g., TWEEN-80).

The immobilized surface can be washed for any amount of time. In some aspects, the immobilized surface is washed for at least about 10 minutes per wash. In some aspects, the immobilized surface is washed for at least about 15 minutes per wash. In some aspects, the immobilized surface is washed for at least about 30 minutes per wash. In some aspects, the immobilized surface is washed for at least about 45 minutes per wash. In some aspects, the immobilized surface is washed for at least about 60 minutes per wash. In some aspects, the immobilized surface is washed for at least about 90 minutes per wash. In some aspects, the immobilized surface is washed for at least about 120 minutes per wash. In some aspects, the immobilized surface is washed for at least about 150 minutes per wash. In some aspects, the immobilized surface is washed for at least about 180 minutes per wash. In some aspects, the immobilized surface is washed overnight.

In some aspects, the immobilized surface is washed at room temperature. In some aspects, the immobilized surface is washed at less than room temperature. In some aspects, the immobilized surface is washed at about 4° C. In some aspects, the immobilized surface is washed at about 37° C.

In some aspects, the immobilized surface is subjected to an agitation during one or more wash. In some aspects, the agitation comprises a rocking, a shaking, a turning, a rotating, a spinning, or any combination thereof. In certain aspects, the immobilized surface is rocked during one or more wash. In some aspects, the immobilized surface is rocked on a plate shaker during one or more wash, wherein the plate shaker is set to a speed of less than about 1000 rpm, less than about 900 rpm, less than about 800 rpm, less than about 700 rpm, less than about 600 rpm, less than about 500 rpm, less than about 450 rpm, less than about 400 rpm, less than about 350 rpm, less than about 300 rpm, less than about 250 rpm, or less than about 200 rpm. In certain aspects, the plate shaker is set to a speed of about 400 rpm. In certain aspects, the plate shaker is set to a speed of about 375 rpm. In certain aspects, the plate shaker is set to a speed of about 350 rpm. In certain aspects, the plate shaker is set to a speed of about 325 rpm. In certain aspects, the plate shaker is set to a speed of about 300 rpm.

In certain aspects, the immobilized surface and/or the sample are protected from light during one or more steps described herein. In some aspects, the immobilized surface and the sample are protected from light during the step wherein the immobilized surface is contacted with the sample. In some aspects, the immobilized surface and the sample are protected from light during the wash following contacting of the immobilized surface and the sample. In some aspects, the immobilized surface and the sample are protected from light during the step wherein the immobilized surface is contacted with the antibody solution. In some aspects, the immobilized surface and the sample are protected from light during the wash following contacting of the immobilized surface and the antibody solution. In some aspects, the immobilized surface and the sample are protected from light at all stages of the methods disclosed herein.

### II.E. Retinoids and Fat-Soluble Vitamins

In some aspects, the liposome, e.g., LNP, comprises one or more retinoid or fat-soluble vitamin, which is displayed on the exterior surface of the liposome, e.g., LNP. LNPs have emerged as a useful vehicle for delivery of various biologically active molecules, including polynucleotides and inhibitory RNAs in particular. Some LNPs have been engineered to comprise one or more retinoid or fat-soluble vitamin on their surface to increase targeting of the LNP, and its cargo, to, e.g., liver cells. Surface displayed retinoids or fat-soluble vitamins on the LNP are able to interact with RBP expressed in liver cells, increasing the localization of the LNP to the liver in vivo.

The methods disclosed herein are capable of detecting and measuring the binding affinity of a liposome, e.g., an LNP, comprising any retinoid or the fat-soluble vitamin, so long as the retinoid or the fat-soluble vitamin is capable of interacting with RBP. As such, any retinoid or fat-soluble vitamin known in the art to interact with RBP, including, but not limited to vitamin A and derivatives and analogs thereof, can be used in the methods disclosed herein. In some aspects, the retinoid or the fat-soluble vitamin comprises vitamin A. In some aspects, the retinoid or the fat-soluble vitamin is selected from the group consisting of retinoic acid, tretinoin, adapalene, 4-hydroxy(phenyl)retinamide (4-HPR), retinyl palmitate, retinal, saturated retinoic acid, and saturated, demethylated retinoic acid.

In certain aspects, the retinoid comprises at least two vitamin A motifs, wherein each vitamin A motif is selected from a vitamin A, a vitamin A analog, and a vitamin A derivative. In some aspects, the at least two vitamin A motifs are connected by a chemical linker. In some aspects, the chemical linker comprises an organic polymer. In certain aspects, the at least two vitamin A motifs are connected by an organic polymer comprising polyethylene glycol. In some aspects, the retinoid or fat-soluble vitamin is selected from the group consisting of retinoid-PEG-retinoid, (retinoid)2-PEG-(retinoid)2, (vitamin A)-PEG2000-(vitamin A), (retinoid)2-bis-amido-PEG-(retinoid)2, and (retinoid)2-Lys-bis-amido-PEG-Lys-(retinoid)2.

In some aspects, the retinoid or the fat-soluble vitamin is synthetic. Various synthetic retinoids, including vitamin A derivatives and analogs, sutiable for the methods disclosed herein are disclosed in U.S. Publication No. 2013/0171240 A1, which is incorporated by reference herein in its entirety. In some aspects, the retinoid or the fat-soluble vitamin comprises a synthetic vitamin A analog disclosed in Table 1. In certain aspects, the retinoid or the fat-soluble vitamin comprises DiVA-PEG-DiVA (N1,N19-bis((S,23E,25E,27E,29E)-16-((2E,4E,6E,8E)-3,7-dimethyl-9-(2,6,6-trimethylcyclo-hex-1-en-1-yl)nona-2,4,6,8-tetraenamido)-24,28-dimethyl-15,22-dioxo-30-(2,6,6-trimethylcyclohexl-en-1-yl)-4,7,10-trioxa-14,21-diazatriaconta-23,25,27,29-tetraen-1-yl)-4,7,10,13,16-pentaoxanonadecane-1,19-diamide).

TABLE 1
Example synthetic Vitamin A Analogs

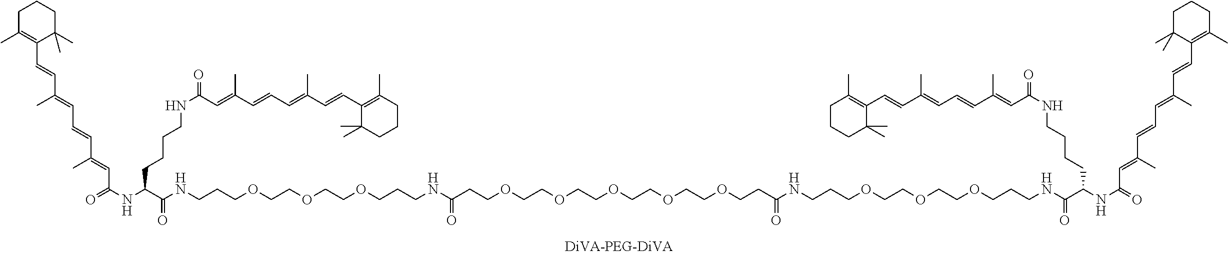

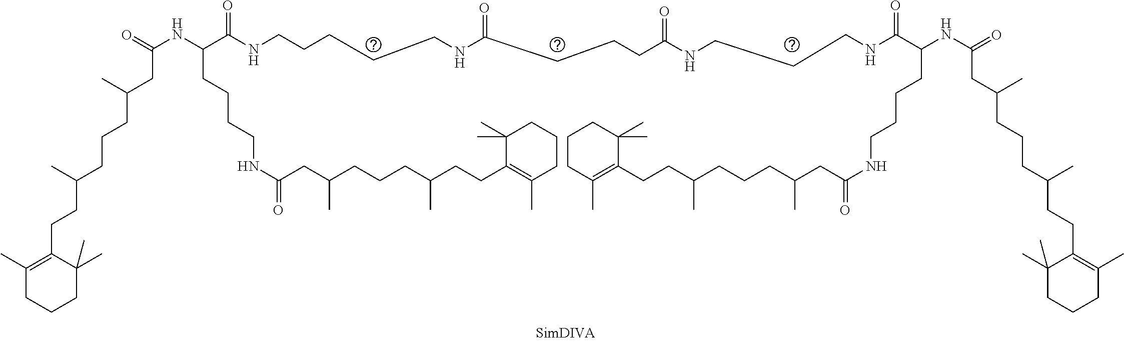

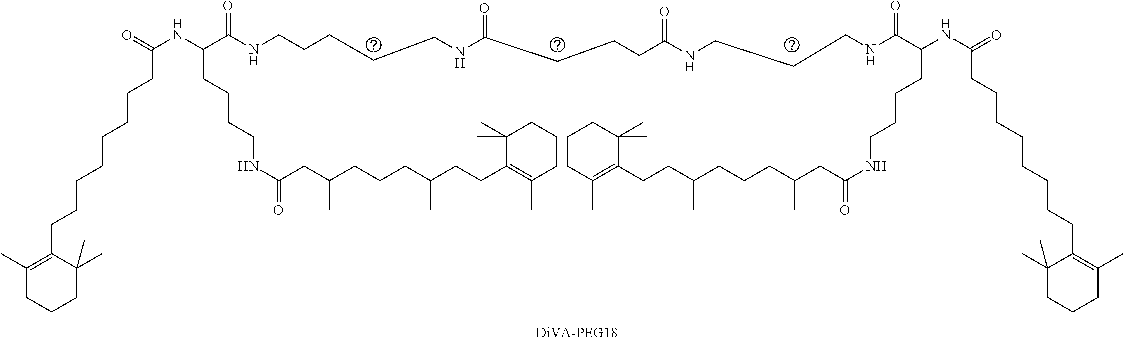

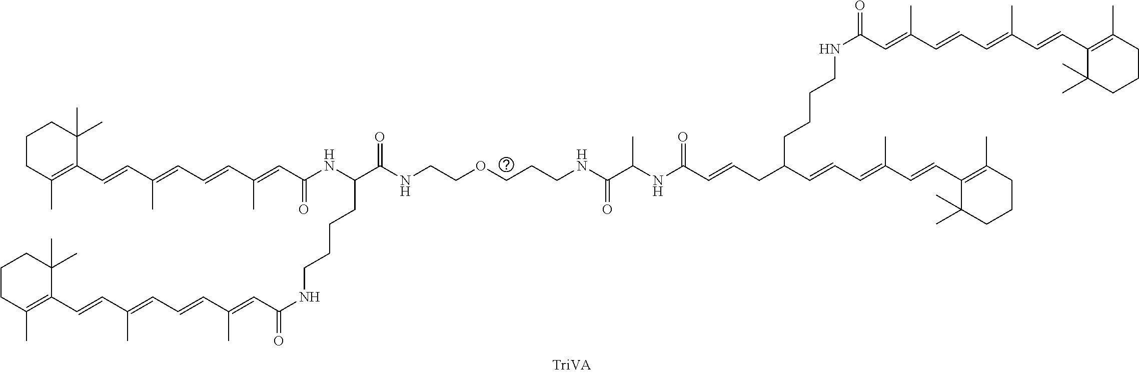 [SMILES: CC1=C(/C=C/C(C)=C/C=C/C(C)=C/C(=O)NCCCCC(/C=C/C=C(C)/C=C/C2=C(C)CCCC2(C)C)C/C=C/C(=O)NC(C)C(=O)NCCCOCCNC(=O)C(CCCCNC(=O)/C=C(C)/C=C/C=C(C)/C=C/C2=C(C)CCCC2(C)C)NC(=O)/C=C(C)/C=C/C=C(C)/C=C/C2=C(C)CCCC2(C)C)C(C)(C)CCC1]

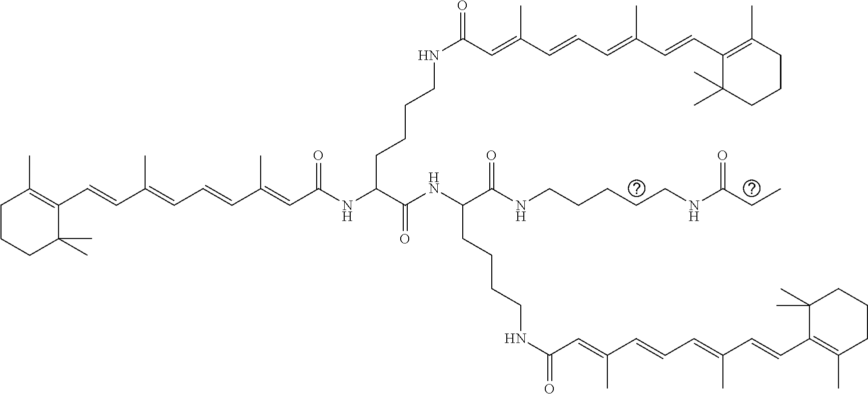 [SMILES: CCC(=O)NCCCCCNC(=O)C(CCCCNC(=O)/C=C(C)/C=C/C=C(C)/C=C/C1=C(C)CCCC1(C)C)NC(=O)C(CCCCNC(=O)/C=C(C)/C=C/C=C(C)/C=C/C1=C(C)CCCC1(C)C)NC(=O)/C=C(C)/C=C/C=C(C)/C=C/C1=C(C)CCCC1(C)C]

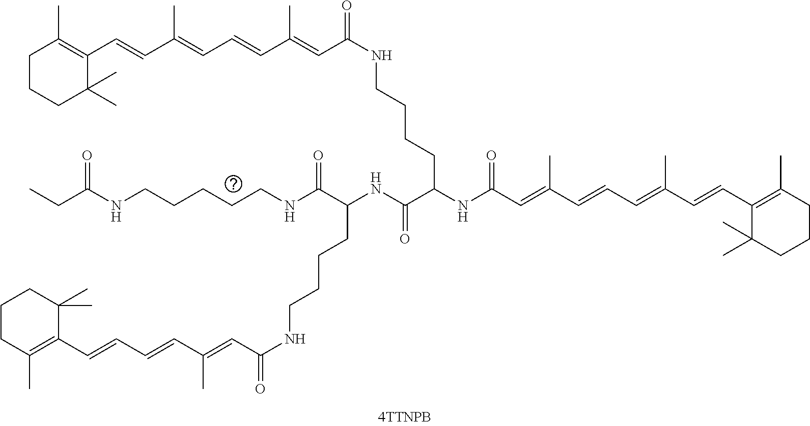 [SMILES: CCC(=O)NCCCCCNC(=O)C(CCCCNC(=O)/C=C(C)/C=C/C=C/C1=C(C)CCCC1(C)C)NC(=O)C(CCCCNC(=O)/C=C(C)/C=C/C=C(C)/C=C/C1=C(C)CCCC1(C)C)NC(=O)/C=C(C)/C=C/C=C(C)/C=C/C1=C(C)CCCC1(C)C]

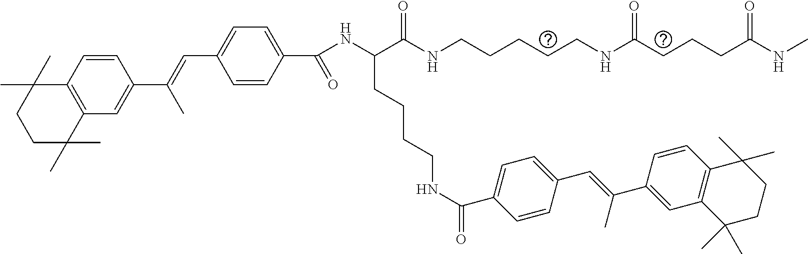

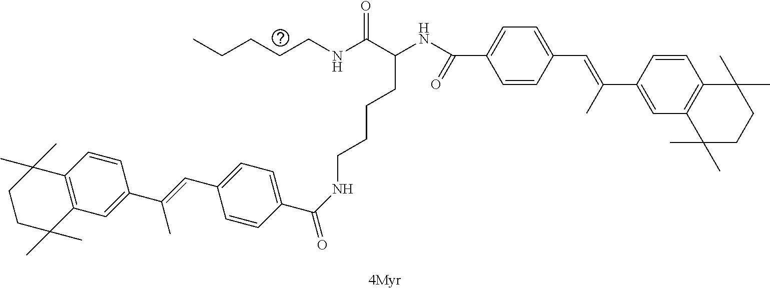

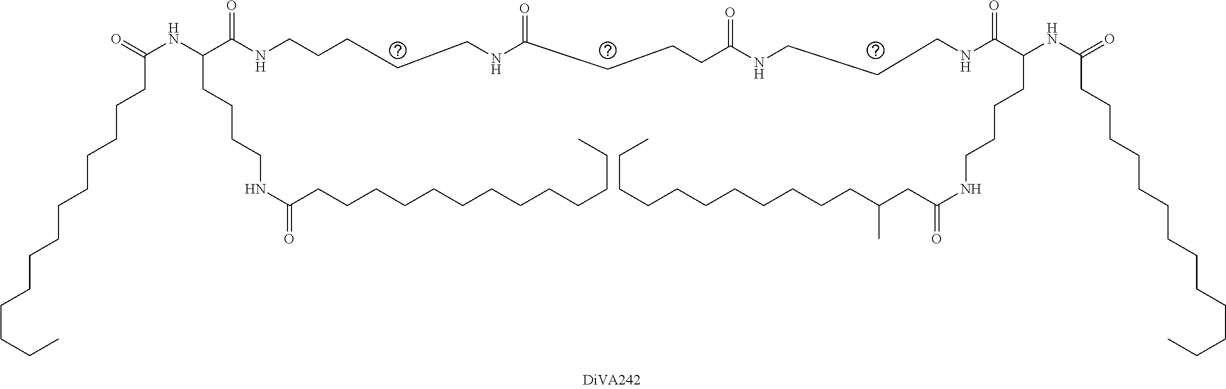 [SMILES: CCCCCCCCCCCCCC(=O)NCCCCC(NC(=O)CCCCCCCCCCCCC)C(=O)NCCCCCNC(=O)CCCC(=O)NCCCNC(=O)C(CCCCNC(=O)CC(C)CCCCCCCCCCC)NC(=O)CCCCCCCCCCCCC]

### II.F. Modified Retinol Binding Proteins

In some aspects, the methods disclosed herein comprise contacting a sample comprising a liposome, e.g., LNP, comprising a surface displayed retinoid or fat soluble vitamin, with a modified RBP associated with an immobilized surface, wherein the modified RBP binds the retinoid or the fat-soluble vitamin. In some aspects, the RBP is modified to enhance the binding affinity of the RBP for the retinoid or the fat-soluble vitamin. In some aspects, the RBP is modified to anchor the RBP to the immobilized surface.

The modified RBP can be anchored to the immobilized surface using any means known in the art. In some aspects, the RBP is chemically linked to the immobilized surface, directly or indirectly. In some aspects, the RBP is modified to comprise a heterologous moiety, wherein the immobilized surface comprises a binding element capable of interacting with the heterologous moiety. In some aspects, the heterologous moiety comprises a ligand, and the immobilized surface comprises a receptor that specifically binds the ligand.

In some aspects, the heterologous moiety comprises biotin. Various biotin-binding partners are known in the art, and any biotin-binding partner can be used in the methods disclosed herein. In certain aspects, the heterologous moiety comprises biotin, and the immobilized surface comprises a binding element selected from the group consisting of avidin, streptavidin, NeutrAvidin, CaptAvidin, and any combination thereof. In certain aspects, the modified RBP is linked to a biotin molecule, and the immobilized surface comprises a streptavidin molecule. In certain aspects, the immobilized surface is coated in streptavidin, avidin, NeutrAvidin, CaptAvidin, or any combination thereof. In some aspects, the immobilized surface is coated in streptavidin. In other aspects, the liposome, e.g., the LNP, does not comprise a Biotin molecule.

Any immobilized surface known in the art can be used in the methods described herein. In some aspects, the immobilized surface is selected from a plate, a bead, a chip, a silica nanoparticle, a carbon nanotube, and any combination thereof. In some aspects, the bead is selected from a glass bead, a metal bead, a magnetic bead, a plastic bead, and any combination thereof.

In certain aspects, the immobilized surface is a plate, wherein the plate is coated with streptavidin, avidin, NeutrAvidin, CaptAvidin, or any combination thereof. In some aspects, the plate is further modified to comprise one or more electrodes, which are associated with the bottom of the plate. In some aspects, the plate comprises at least one well, wherein the at least one well comprises an electrode surface, which absorbs capture reagent. In some aspects, the plate has a hydrophobic surface. In some aspects, the plate has a hydrophilic surface. In certain aspects, the plate is a SECTOR™ plate or a QUICKPLEX® plate (Meso Scale Diagnostics).

In some aspects, the immobilized surface comprises a sensor chip (e.g., a Biacore chip).

### II.G. Immobilized Surfaces

In some aspects, the methods disclosed herein comprise contacting a sample comprising a liposome, e.g., LNP, comprising a surface displayed retinoid or fat soluble vitamin, with a modified RBP associated with an immobilized surface, wherein the modified RBP binds the retinoid or the fat-soluble vitamin. The method described herein can use an immobilized surface suitable for bio-detection assays. Non-liming examples of immobilized surfaces usable in the methods disclosed herein include, but are not limited to, a plate, a magnetic bead, a chip, a column, and any combination thereof.

In some aspects, the immobilized surface comprises a plate. In some aspects, the plate is a carbon coated plate. In some aspects, the plate comprises one or more electrodes. In some aspects, the one or more electrodes are capable of facilitating electrochemiluminescence in a well of the plate. In certain aspects, the plate is a SECTOR plate (MSD). In some aspects, the plate is a QuickPlex plate (MSD). In some aspects, the plate is a SECTOR PR plate (MSD).

In some aspects, the immobilized surface comprises a bead. In some aspects, the bead is selected from a magnetic bead, a glass bead, a silicate bead, a plastic bead, and any combination thereof. In certain aspects, the immobilized surface comprises a magnetic bead.

In some aspects, the immobilized surface comprises a chip. In certain aspects, the immobilized surface comprises a sensory chip. In certain aspects, the immobilized surface comprises a carboxy methylated dextran sensor chip. In certain aspects, the immobilized surface comprises a Biacore chip.

### II.H. Biologically Active Moieties

Certain aspects of the present disclosure are directed to methods of assaying the binding affinity of a liposome, e.g., an LNP, comprising a surface displayed retinoid or fat-soluble vitamin, to RBP. In some aspects, the liposome, e.g., LNP, comprises one or more biologically active moiety. The biologically active moiety can be associated with the lipid layer of the liposome, e.g., LNP. In some aspects, the biologically active moiety is associated with an exterior surface of the lipid layer of the liposome, e.g., LNP. In some aspects, the biologically active moiety is associated with a luminal surface of the lipid layer of the liposome, e.g., LNP. In some aspects, the biologically active moiety is present in the lumen of the liposome, e.g., LNP, without being associated with the lipid layer of the liposome, e.g., LNP.

In some aspects, the biologically active moiety is anchored to the lipid layer of the liposome, e.g., LNP. The biologically active moiety can be anchored to the lipid layer of the liposome, e.g., LNP, by any mechanism known in the art. In some aspects, the biologically active moiety is anchored to the lipid layer of the liposome, e.g., LNP, by a covalent bond. In some aspects, the biologically active moiety is anchored to the lipid layer of the liposome, e.g., LNP, by a linker. In some aspects, the linker comprises a peptide linker, a chemical linker, or a combination thereof. In some aspects, the biologically active moiety comprises a hydrophobic tag, e.g., a cholesterol tag, which associates with the lipid layer of the liposome, e.g., LNP, and anchors the biologically active moiety to the lipid layer. In some aspects, the biologically active moiety associates with the lipid layer through one or more non-covalent interactions. In certain aspects, the biologically active moiety associates with the lipid layer through an electrostatic interaction.

In certain aspects, the biologically active moiety comprises a polynucleotide. In some aspects, the polynucleotide is an RNA. In certain aspects, the RNA is selected from the group consisting of an mRNA, a miRNA, an siRNA, a dsRNA, and any combination thereof. In some aspects, the biologically active moiety comprises an siRNA that reduces expression of heat shock protein 47 (HSP47). In some aspects, the biologically active moiety comprises an HSP47-specific siRNA. In particular aspects, the HSP46-specific siRNA comprises a passenger polynucleotide and a guide polynucleotide. In some aspects, the passenger polynucleotide comprises the nucleotide sequence set forth in SEQ ID NO: 1 (5′-iAb-GAGACACAUGGGUGcuaua-C3-Pi-3′). In some aspects, the guide polynucleotide comprises the nucleotide sequence set forth in SEQ ID NO: 2 (3′-C3-C3-CUCUGUGUACCCaCGAUAU-5′).

In some aspects, the polynucleotide comprises an antisense oligonucleotide. In some aspects, the biologically active moiety comprises a DNA. In some aspects, the DNA is selected from an ssDNA, a dsDNA, a plasmid, a fragment thereof, or any combination thereof.

In some aspects, the biologically active molecule comprises a polypeptide. In some aspects, the polypeptide is selected from an enzyme, a growth factor, a cytokine, a chemokine, a ligand, a receptor, a hormone, an antibody, an antigen, a fragment thereof, or any combination thereof. In some aspects, the polypeptide comprises a nuclease. In some aspects, the nuclease is selected from the group consisting of a Cas9 nuclease, a TALN nuclease, a zinc finger nuclease, and any combination thereof.

In some aspects, the biologically active moiety comprises a small molecule, e.g., a chemical compound.

In some aspects, the liposome, e.g., LNP, is further administered to a subject in need thereof. Various liposomes disclosed herein are capable of treating preventing or treating a disease or condition in a subject in need thereof. In particular, liposomes, e.g., LNPs, comprising a surface displayed retinoid or fat soluble molecule capable of interacting with biotin are capable of targeting liver cells in a subject. As such, certain aspects of the present disclosure are directed to methods of assaying the targeting ability of liposomes, e.g., LNPs, to liver cells, wherein the liposomes, e.g., LNPs, are further administered to a subject in need thereof to treat a disease or condition affecting the liver. In some aspects, the disease or condition affecting the liver is selected from the group consisting of hepatic cirrhosis, liver fibrosis, nonalcoholic steatohepatitis (NASH), alcoholic steatohepatitis, primary sclerosing cholangitis, primary biliary cirrhosis, and any combination thereof.

In other aspects, the liposome is administered to a subject in need thereof to treat a disease or condition comprising an organ fibrosis. In some aspects, the organ fibrosis is selected from the group consisting of liver fibrosis, lung fibrosis, a glial scar, arterial stiffness, arthrofibrosis, Crohn's disease, Dupuytren's contracture, keloid formation, mediastinal fibrosis, myelofibrosis, peyronie's disease, nephrogenic systemic fibrosis, progressive massive fibrosis, retroperitoneal fibrosis, scleroderma/systemic sclerosis, adhesive capsulitis, and any combination thereof.

### III. Kits

Some aspects of the present disclosure are directed to a kit comprising a modified RBP disclosed herein. In some aspects, the kit further comprises an immobilized surface disclosed herein. In some aspects, the kit further comprises an antibody or an antigen-binding fragment thereof disclosed herein. In certain aspects, the kit comprises an anti-PEG antibody disclosed herein. In some aspects, the kit further comprises a blocking solution disclosed herein. In some aspects, the kit further comprises one or more wash buffer disclosed herein. In some aspects, the kit further comprises an antibody solution disclosed herein. In some aspects, the kit further comprises instructions to assay the binding affinity of a liposome, e.g., an LNP, to an RBP according to a method disclosed herein.

### EXAMPLES

### Example 1

### MSD RBP Binding Assay

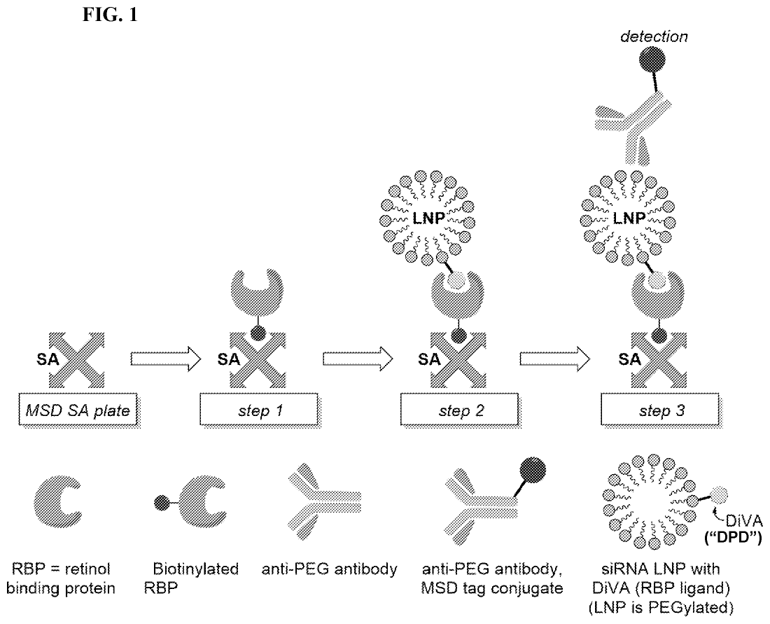

An immunoassay was developed for measuring RBP binding to lipid nanoparticles (LNPs) having surface exposed DiVA-PEG-DiVA (DPD), e.g., the therapeutic LNP drug product. The immunoassay employed a single-plex sandwich immunoassay format in carbon electrode microtiter plates (streptavidin coated), and ECL detection measured on an MSD Sector Imager plate reader (Meso Scale Diagnostics, LLC.) (FIG. 1). In this assay the streptavidin coated plates were first incubated with biotin-conjugated native human Retinol-Binding Protein (RBP4). LNP drug product control and test samples were then incubated, followed by sequential addition of SULFO-TAG conjugated anti-PEG antibody (against Methoxy group) and ECL read buffer. Plates were read in the MSD plate reader where an electrical potential was applied resulting in measurable ECL emission. The amount of LNP drug product bound to the plate through DPD-RBP binding was directly proportional to the amount of light emitted. Results were reported as percent binding relative to reference.

### RiboGreen Assay

The integrity of LNP drug product was measured by a fluorescence-based assay method using RiboGreen® RNA reagent, a fluorescent nucleic acid stain. A series of siRNA standard solutions were prepared in PBS buffer containing various level of Tween20 with siRNA concentrations at 0.2, 1.0 and 2.0 μg/mL. The sample solutions for measurement of unencapsulated siRNA (free RNA sample solution) were prepared by diluting LNP suspension in PBS buffer with each level of Tween20 to have siRNA concentration at approximately 20 μg/mL. The prepared free siRNA sample solutions were further diluted in PBS buffer with 1% Triton to make a sample solution to measure the total amount of both encapsulated and non-encapsulated as internal control. Then the siRNA standard solutions and LNP sample solutions were mixed with TE buffer containing 0.5% RiboGreen® RNA reagent in a 96-well flat-bottom tissue culture plate at 1:1 ratio. The fluorescent responses from each well were measured using the SpecMax M2 fluorimeter at 485 nm (excitation) and 528 nm (emission). The amount of free siRNA were calculated as percentage of total siRNA in sample solution and the variation of which indicates the change of LNP integrity. Experiments are documented in notebook A 1D8F-025.

### Results and Discussion

### Titration Curve

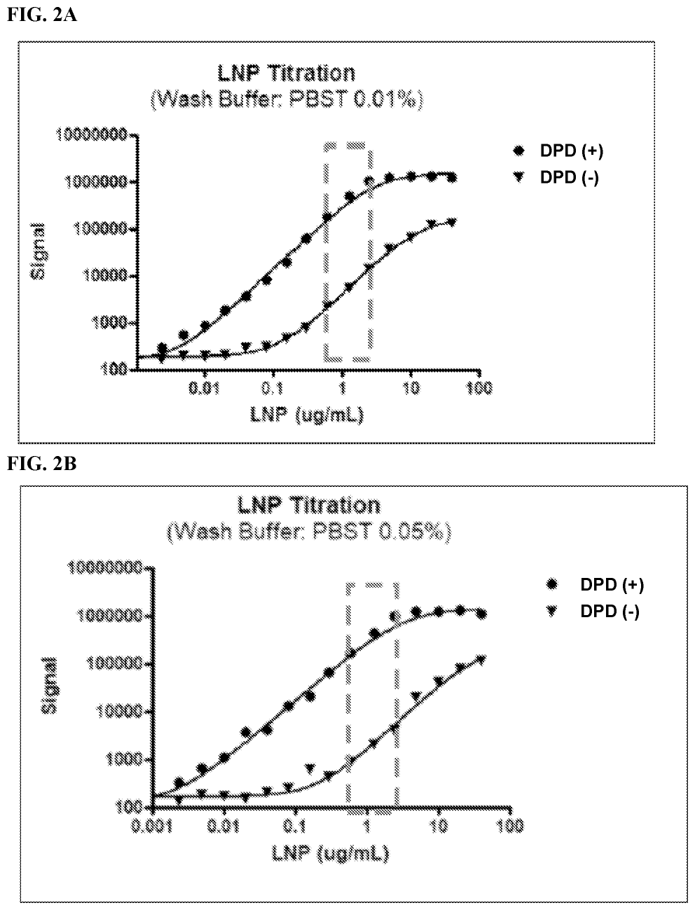

The LNP titration curve was developed by exploring a DPD (+) and a DPD (−) concentration range of 0-50 μg/mL using 0.01% (FIG. 2A) or 0.05% (FIG. 2B) PBS-Tween20 (PBST) wash buffers. The resulting titration curve showed a linear range between 0.1-2.5 μg/mL. The apparent signal-to-noise (S/N) ratio of the positive and negative signal (or P/N ratio) was highest from 0.3-2.5 μg/mL. With PBST 0.05% the DPD (−) concentration is 1% of the DPD (+) concentration. While 0.05% PBST presented better separation compared to 0.01% PBST, it is possible that the presence of Tween20 could disrupt the LNP. In conclusion, this experiment showed that a sample testing concentration range from 1-2 μg/mL provided the best recovery and P/N ratios. The potential of LNP disruption by Tween20 was evaluated further during development and is reported below.

### LNP Reconstitution Stability and Assay Robustness

LNP samples were reconstituted and analyzed on days 0, 1, 2, 6, 10, 14, 15, 16, 20, 21, 24, 29, and 35 to check for reconstitution stability and reproducibility (Tables 4-9). The intra-plate signal reproducibility for both 0.01% and 0.05% PBST was acceptable with a % CV<15%. The inter-day signal variability was much higher, at 28-44% CV. Using 0.01% PBST as the wash buffer, day 21 signals were within 70-80% of signals at day 0, even with proper storage away from light. Interestingly, with the use of 0.05% PBST (and not 0.01% PBST) there appeared to be an increase in signal over time suggesting possible disruption of the LNP.

TABLE 2
Titration Curve Signal of LNP drug product (16NIA01-v2) Ran in Different Days (0.01% PBST)

| Titration | | | | | | | | | | |
|---|---|---|---|---|---|---|---|---|---|---|
| Curve Signal - | DPD (+) 16NIA01-v2 | | | | | | | | | |
| 0.01% PBST | Run 1 | Run 2 | Run 3 | Run 4 | Run 5 | Run 6 | Run 7 | | | |
| Date/Days | Feb. 2, | Feb. 27, | Feb. 28, | Mar. 4, | Mar. 8, | Mar. 13, | Mar. 19, | | | Intra-plate |
| after | 2019 | 2019 | 2019 | 2019 | 2019 | 2019 | 2019 | Inter-plate (n = 7) | | Signal CV |
| Reconstitution | Day 0 | Day 1 | Day 2 | Day 6 | Day 10 | Day 15 | Day 21 | Signal | Signal | average |
| LNP (μg/mL) | Average Signal (n=2) | | | | | | | Average | CV | (n = 7) |
| 8 | 834,292 | 1,298,304 | 991,350 | 612,449 | 852,354 | 534,890 | 517,069 | 805,815 | 35% | 4.2 |
| 2 | 407,403 | 791,277 | 611,195 | 361,843 | 574,911 | 334,569 | 374,242 | 493,634 | 34% | 4.4 |
| 0.5 | 73,750 | 147,284 | 118,173 | 63,107 | 86,949 | 55,166 | 64,622 | 87,007 | 39% | 1.6 |
| 0.1 | 14,911 | 20,930 | 19,356 | 9,909 | 13,125 | 6,523 | 8,759 | 13,359 | 40% | 9.6 |
| 0.03 | 2,348 | 4,056 | 3,687 | 2,089 | 3,044 | 1,089 | 1,413 | 2,532 | 44% | 7.0 |
| 0.008 | 587 | 981 | 1,073 | 561 | 882 | 366 | 375 | 689 | 42% | 6.3 |
| 0.0020 | 251 | 313 | 357 | 233 | 374 | 170 | 140 | 263 | 34% | 3.2 |
| 0 | 122 | 140 | 118 | 87 | 115 | 92 | 66 | 106 | 24% | 6.1 |

TABLE 3
Corrected Concentration Measurement of LNP drug product (16NIA01-v2)
Reconstituted for Different Days (0- 21 days) (0.01% PBST)

| DPD Positive LNPs - | | | | | | | | | | | |
|---|---|---|---|---|---|---|---|---|---|---|---|
| Sample Set 2 (0-21 | | | | | | | | | | | |
| days post | | | | | | | | | | | |
| reconstitution, 0.01% | | | | | | | | | | | |
| PBST) | | DPD (+) 16NIA01-v2 | | | | | | | | | |
| LNP | Nominal | Dilution corrected concentration (mg/mL) | | | | | | | | | |
| Testing | Stock | n = 2 | | | | | | | | | |
| Conc | Conc | Day | Day | Day | Day | Day | Day | Day | Average | Conc | % |
| (μg/mL) | (mg/mL) | 0 | 1 | 2 | 6 | 10 | 15 | 21 | Conc | CV | Recovery |
| 2 | 3.0 | 4.49 | 5.24 | 4.63 | 4.53 | 6.33 | 4.42 | 5.54 | 5.03 | 14% | 168% |
| 1 | 3.0 | 3.19 | 3.67 | 4.22 | 3.24 | 4.34 | 3.21 | 3.52 | 3.63 | 13% | 121% |
| 0.5 | 3.0 | 2.64 | 3.08 | 3.16 | 2.55 | 2.96 | 2.86 | 2.63 | 2.84 | 8% | 95% |
| 0.25 | 3.0 | 2.38 | 2.74 | 2.55 | 2.34 | 2.44 | 2.54 | 2.26 | 2.46 | 6% | 82% |

TABLE 4
Corrected Concentration Measurement of LNP drug product (16NIA01-v1)
Reconstituted for Different Days (14-35 days) (0.01% PBST)

| DPD Positive LNPs - | | | | | | | | | | | |
|---|---|---|---|---|---|---|---|---|---|---|---|
| Sample Set 1 (14-35 | | | | | | | | | | | |
| days post | | | | | | | | | | | |
| reconstitution, 0.01% | | | | | | | | | | | |
| PBST) | | DPD (+) 16NIA01-v1 | | | | | | | | | |
| LNP | Nominal | Dilution corrected concentration (mg/mL) | | | | | | | | | |
| Testing | Stock | n=2 | | | | | | | | | |
| Conc | Conc | Day | Day | Day | Day | Day | Day | Day | Average | Conc | % |
| (μg/mL) | (mg/mL) | 14 | 15 | 16 | 20 | 24 | 29 | 35 | Conc | CV | Recovery |
| 2 | 3.0 | 7.94 | 6.14 | 5.10 | 5.45 | 6.73 | 4.57 | 5.41 | 5.91 | 19% | 197% |
| 1 | 3.0 | 4.72 | 4.46 | 4.19 | 4.44 | 4.55 | 3.41 | 3.75 | 4.21 | 11% | 140% |
| 0.5 | 3.0 | 3.55 | 3.59 | 3.28 | 3.13 | 3.01 | 2.96 | 2.81 | 3.19 | 9% | 106% |
| 0.25 | 3.0 | 3.07 | 3.13 | 2.68 | 2.56 | 2.54 | 2.58 | 2.33 | 2.70 | 11% | 90% |

TABLE 5
Titration Curve Signal of LNP drug product (16NIA01-v2) Ran in Different Days (0.05% PBST)

| Titration | | | | | | | | | | |
|---|---|---|---|---|---|---|---|---|---|---|
| Curve Signal - | DPD (+) 16NIA01-v2 | | | | | | | | | |
| 0.05% PBST | Run 1 | Run 2 | Run 3 | Run 4 | Run 5 | Run 6 | Run 7 | | | |
| Date/Days | Feb. 26, | Feb. 27, | Feb. 28, | Mar. 4, | Mar. 8, | Mar. 13, | Mar. 19, | | | Intra-plate |
| after | 2019 | 2019 | 2019 | 2019 | 2019 | 2019 | 2019 | Inter-plate (n = 7) | | Signal CV |
| Reconstitution | Day 0 | Day 1 | Day 2 | Day 6 | Day 10 | Day 15 | Day 21 | Signal | Signal | average |
| LNP (μg/mL) | Average Signal (n=2) | | | | | | | Average | CV | (n = 7) |
| 8 | 598,836 | 734,404 | 854,276 | 921,464 | 1,272,722 | 601,711 | 934,439 | 845,407 | 28% | 3.9 |
| 2 | 249,771 | 666,888 | 585,829 | 789,184 | 983,032 | 556,359 | 939,947 | 681,573 | 37% | 5.5 |
| 0.5 | 39,024 | 99,358 | 81,233 | 119,868 | 126,087 | 83,164 | 188,438 | 105,310 | 44% | 6.6 |
| 0.1 | 7,610 | 16,296 | 11,960 | 19,234 | 18,833 | 11,468 | 26,343 | 15,963 | 39% | 14.2 |
| 0.03 | 1,722 | 3,789 | 3,747 | 4,832 | 5,567 | 2,759 | 5,549 | 3,995 | 36% | 13.2 |
| 0.008 | 482 | 1,026 | 864 | 1,359 | 1,282 | 759 | 1,643 | 1,059 | 37% | 6.5 |
| 0.0020 | 199 | 300 | 367 | 473 | 484 | 311 | 438 | 367 | 29% | 5.5 |
| 0 | 90 | 110 | 102 | 118 | 118 | 111 | 98 | 107 | 10% | 6.7 |

TABLE 6
Corrected Concentration Measurement of LNP drug product (16NIA01-v2) Reconstituted for Different Days
(0-21 days) (0.05% PBST)

| DPD Positive LNPs - | | | | | | | | | | | |
|---|---|---|---|---|---|---|---|---|---|---|---|
| Sample Set 2 (0-21 | | | | | | | | | | | |
| days post reconstitution, | | | | | | | | | | | |
| 0.05% PBST) | | DPD (+) 16NIA01-v2 | | | | | | | | | |
| LNP Testing | Nominal Stock | Dilution corrected concentration (mg/mL) n = 2 | | | | | | | Average | Conc | % |
| Conc (μg/mL) | Conc (mg/mL) | Day 0 | Day 1 | Day 2 | Day 6 | Day 10 | Day 15 | Day 21 | Conc | CV | Recovery |
| 2 | 3.0 | 4.88 | 6.38 | 5.08 | 6.16 | 5.90 | 8.67 | 8.36 | 6.49 | 23% | 216% |
| 1 | 3.0 | 3.99 | 4.17 | 4.01 | 4.23 | 4.00 | 4.90 | 4.67 | 4.28 | 8% | 143% |
| 0.5 | 3.0 | 3.08 | 2.91 | 3.26 | 3.13 | 2.87 | 3.44 | 3.06 | 3.11 | 6% | 104% |
| 0.25 | 3.0 | 2.66 | 2.97 | 2.65 | 2.57 | 2.18 | 2.87 | 2.41 | 2.61 | 10% | 87% |

TABLE 7
Corrected Concentration Measurement of LNP drug product (16NIA01-v1) Reconstituted for Different Days
(14-35 days) (0.05% PBST)

| DPD Positive LNPs - | | | | | | | | | | | |
|---|---|---|---|---|---|---|---|---|---|---|---|
| Sample Set 1 (14-35 | | | | | | | | | | | |
| days post reconstitution, | | DPD (+) 16NIA01-v1 | | | | | | | | | |
| 0.05% PBST) | | Dilution corrected concentration (mg/mL) n = 2 | | | | | | | | | |
| LNP Testing | Nominal Stock | Day | Day | Day | Day | Day | Day | Day | Average | Conc | % |
| Conc (μg/mL) | Conc (mg/mL) | 14 | 15 | 16 | 20 | 24 | 29 | 35 | Conc | CV | Recovery |
| 2 | 3.0 | 8.93 | 9.77 | 7.49 | 10.25 | 6.68 | 14.89 | 10.29 | 9.76 | 27% | 325% |
| 1 | 3.0 | 6.64 | 5.62 | 4.96 | 6.09 | 4.98 | 5.57 | 4.94 | 5.54 | 12% | 185% |
| 0.5 | 3.0 | 5.06 | 3.60 | 3.85 | 4.23 | 3.18 | 4.07 | 3.32 | 3.90 | 16% | 130% |
| 0.25 | 3.0 | 4.29 | 3.50 | 3.47 | 3.14 | 2.47 | 2.95 | 2.49 | 3.19 | 20% | 106% |

LNPs were also tested at concentrations ranging between 0.25-2 μg/mL up to 35 days using both 0.01% and 0.05% PBST. With both PBST wash buffers, measurements were accurate at a sample concentration ranging from 0.25-0.5 μg/mL, although better recovery was observed at 0.5 μg/mL. These data also indicated that the sample was stable for 3-5 weeks after reconstitution.

Despite high signal variability across days (30-40%), results showed excellent reproducibility and accuracy across 21 days. Samples were stable for 3-5 weeks after reconstitution. In general, 0.05% and 0.01% PB ST showed similar results. However, 0.05% PBST provided less accurate results when used with older samples that were reconstituted for a longer period of time, again suggesting high probability of LNP disruption from Tween20. In conclusion, the more suitable wash buffer was deemed 0.01% PB ST. Additionally, a working concentration of 0.25-0.5 μg/mL provided the best recovery and reproducibility within the linear range of the titration curve.

### Assay Parameter Robustness

Assay robustness (Table 8) was evaluated to assess overall method variation. Test samples were diluted to 0.25-2.0 μg/mL and run in the assay using either 0.01% or 0.05% PBST wash buffer condition. When 0.01% PBST was used as wash buffer and test samples diluted to 0.25-0.5 μg/mL were analyzed, no significant impact on RBP binding affinity was observed in the assay (Table 8).

TABLE 8
Robustness Evaluation.

| Tested Conditions | Impact on Assays |
|---|---|
| Exposure to white light | No significant impact. |
| (0, 30, 60, min) | |
| Storage at low concentrations | No impact at 0.25-0.5 |
| (0.25-2 μg/mL) | μg/mL working |
| | concentrations when tested |
| | with 0.01% PBST. |
| Storage at room temperature | No significant impact. |
| (protected from light) for 3-18 hours | |
| Lower plate shaking | No significance impact when |
| speed (300 rpm) | tested with 0.01% PBST. |
| Higher incubation temperature | No significance impact when |
| (37° C.) | tested with 0.01% PBST. |
| Longer sample incubation time | No impact at 0.25-0.5 μg/mL |
| (overnight) | working concentrations when |
| | tested with 0.01% PBST. |

DPD is a light sensitive molecule due to the vitamin A moieties in its structure, hence, the whole assay procedure is protected from light. Method performance with and without the presence of artificial white light was evaluated. LNP samples were exposed to artificial white light in a time course (30 minutes and 60 minutes). The relative % RBP binding of light exposed samples (30 or 60 minutes) were calculated by comparing the RBP binding of the tested sample to the control sample (0 minute). No significant difference in relative % RBP binding of light exposed LNP samples was observed up to 60 minutes exposure when compared to the control, T0 (Table 9).

TABLE 9
% RBP Binding Affinity of
Samples Exposed to Artificial White Light.

| Wash | Diluted | 0 min | 30 min | 60 min |
|---|---|---|---|---|
| Buffer | LNP | (% of 0 min, | (% of | (% of |
| Condition | (μg/mL) | as control) | control) | control) |
| 0.01% PBST | 2 | 100 | 91 | 93 |
| | 1 | 100 | 95 | 99 |
| | 0.5 | 100 | 90 | 102 |
| | 0.25 | 100 | 96 | 96 |
| 0.05% PBST | 2 | 100 | 90 | 94 |
| | 1 | 100 | 94 | 97 |
| | 0.5 | 100 | 96 | 98 |
| | 0.25 | 100 | 81 | 88 |

The LNP samples were diluted to target concentrations (0.25-2.0 μg/mL) immediately before testing. The stabilities of diluted LNP samples were monitored up to 23 hours at 4° C., or tested immediately. Results were calculated by comparing RBP binding of experimental samples to the control samples (freshly diluted). As shown in Table 10, if 0.01% PBST wash buffer was utilized, the relative binding of the experimental samples were comparable to freshly diluted samples, when at a concentration of 0.25-1 μg/mL. However, when the assay used 0.05% PBST wash buffer, the relative RBP binding of the experimental samples were no longer comparable with freshly diluted samples. In conclusion, when test samples are diluted to 0.25-1 μg/mL, they are stable to be stored at 4° C. for up to 23 hours before the assay, and 0.01% PB ST wash buffer is preferrable in the assay.

TABLE 10
% RBP Binding Affinity of Samples Stored at Low
Concentration (4° C. for up to 23 hours)

| Wash Buffer | Diluted LNP | 0 hour (% of 0 | 23 hour |
|---|---|---|---|
| Condition | (μg/mL) | min, as control) | (% of control) |
| 0.01% PBST | 2 | 100 | 410 |
| | 1 | 100 | 123 |
| | 0.5 | 100 | 119 |
| | 0.25 | 100 | 101 |
| 0.05% PBST | 2 | 100 | 361 |
| | 1 | 100 | 176 |
| | 0.5 | 100 | 142 |
| | 0.25 | 100 | 101 |

The LNP drug product samples were typically stored at 4° C. after reconstitution. Here we evaluated the stability of reconstituted LNP drug product samples stored at room temperature in a time course study (3 hours and 18 hours). The relative % RBP binding of room temperature stored samples (3 or 18 hours) were calculated by comparing the RBP binding of the tested sample to the control samples (0 hour). No significant difference in the relative % RBP binding was observed for LNP drug product samples stored at room temperature for up to 18 hours (Table 11). In conclusion, LNP drug product samples were stable at room temperature for up to 18 hours.

TABLE 11
% RBP Binding Affinity of Samples Stored at Room Temperature

| Wash | Diluted | 0 hour (% of | 3 hour | 18 hour |
|---|---|---|---|---|
| Buffer | LNP | 0 hour, as | (% of | (% of |
| Condition | (μg/mL) | control) | control) | control) |
| 0.01% PBST | 2 | 100 | 87 | 92 |
| | 1 | 100 | 86 | 90 |
| | 0.5 | 100 | 85 | 85 |
| | 0.25 | 100 | 89 | 90 |
| 0.05% PBST | 2 | 100 | 74 | 80 |
| | 1 | 100 | 75 | 82 |
| | 0.5 | 100 | 83 | 87 |
| | 0.25 | 100 | 84 | 91 |

The RBP binding assay can use 700 rpm shaking speed and room temperature for sample incubation. Here we evaluated the impact of lower shaking speed (300 rpm) or higher incubation temperature (37° C.) during assay procedure, while the other parameters remained the same. When the assay used 0.01% PBST wash buffer, lower shaking speed or higher incubation temperature did not affect the relative % RBP binding compared with the control samples. However, when the assay used 0.05% PBST wash buffer, the results were less accurate and the relative % RBP binding was less comparable (Table 12). In conclusion, the change of shaking speed (700 rpm to 300 rpm) or incubation temperature (room temperature to 37° C.) are not significantly impactful on the MSD RBP binding assay, if 0.01% PBST wash buffer is utilized in the assay.

TABLE 12
% RBP Binding Affinity of Samples Incubated at Lower
Shaking Speed or Higher Temperature

| | | Control, 700 | 300 rpm, | 700 rpm, |
|---|---|---|---|---|
| Wash | Diluted | rpm, room | room | 37° C. |
| Buffer | LNP | temperature | temperature | (% of |
| Condition | (μg/mL) | (% of itself) | (% of control) | control) |
| 0.01% PBST | 2 | 100 | 113 | 95 |
| | 1 | 100 | 115 | 127 |
| | 0.5 | 100 | 106 | 109 |
| | 0.25 | 100 | 117 | 91 |
| 0.05% PBST | 2 | 100 | 47 | 37 |
| | 1 | 100 | 67 | 66 |
| | 0.5 | 100 | 78 | 75 |
| | 0.25 | 100 | 93 | 72 |

In a typical RBP binding assay, the LNP samples were incubated on RBP-conjugated streptavidin plates for one hour. Here we evaluated assay robustness at longer incubation time (overnight). The relative % RBP binding of samples incubated overnight were calculated by comparing the RBP binding of the experimental samples to the control samples (one hour). When the assay used 0.01% PB ST wash buffer, LNP samples (diluted to 0.25-0.5 μg/mL) that were incubated overnight on the plate remained comparable for relative % RBP binding compared with control samples. However, when the assay used higher LNP dilution concentration or 0.05% PBST wash buffer, results were less robust and relative % RBP binding was not comparable (Table 13). These data show that longer incubation time (from one hour to overnight) doesn't have significant impact on the MSD RBP binding assay if 0.01% PBST wash buffer is utilized and test samples are diluted to 0.25-0.5 μg/mL in the assay.

TABLE 13
% RBP Binding Affinity of
Samples Incubated at Longer Incubation Time

| Wash | Diluted | | Overnight |
|---|---|---|---|
| Buffer | LNP | 1 hour Incubation | Incubation |
| Condition | (μg/mL) | (% of itself, as control) | (% of control) |
| 0.01% PBST | 2 | 100 | 194 |
| | 1 | 100 | 330 |
| | 0.5 | 100 | 134 |
| | 0.25 | 100 | 108 |
| 0.05% PBST | 2 | 100 | 103 |
| | 1 | 100 | 212 |
| | 0.5 | 100 | 370 |
| | 0.25 | 100 | 357 |

### Sample Testing Results

The variability of LNP drug product manufacturing process, material properties (i.e. excipient purity), and/or isolated specific material properties of LNPs (e.g. particle size, LNP structure, etc.) could affect LNP bio-performance (which includes RBP binding affinity). In this experiment, three LNP drug product DoE samples with known atypical particle characteristic (e.g., larger particle size) were analyzed in the assay for relative % RBP binding. As shown in Table 14, all three of the DoE samples showed lower relative % RBP binding compared with the control sample, with both wash buffers. Interestingly, sample lot #13, which had the larger particle size and wider particle distribution, displayed the lowest relative % RBP binding.

TABLE 14
% RBP Binding Affinity of LINP Samples
with Atypical Particle Characteristics

| | | | Dilution | |
|---|---|---|---|---|
| Wash | | Diluted | Corrected | |
| Buffer | Sample | LNP | Conc. | % RBP |
| Condition | Lot# | (μg/mL) | (mg/mL) | Binding |
| 0.01% PBST | 1 | 2 | 3.9 | 129 |
| | | 1 | 2.7 | 90 |
| | | 0.5 | 1.8 | 59 |
| | 13 | 2 | 0.6 | 19 |
| | | 1 | 0.6 | 20 |
| | | 0.5 | 0.6 | 21 |
| | 23 | 2 | 2.6 | 85 |
| | | 1 | 1.8 | 60 |
| | | 0.5 | 1.3 | 42 |
| 0.05% PBST | 1 | 2 | 3.5 | 115 |
| | | 1 | 2.8 | 92 |
| | | 0.5 | 1.4 | 45 |
| | 13 | 2 | 0.8 | 27 |
| | | 1 | 0.8 | 27 |
| | | 0.5 | 0.9 | 30 |
| | 23 | 2 | 2.5 | 84 |
| | | 1 | 1.9 | 63 |
| | | 0.5 | 1.5 | 49 |

### LNP Integrity Comparison Between 0.01% and 0.05% PBS-Tween20

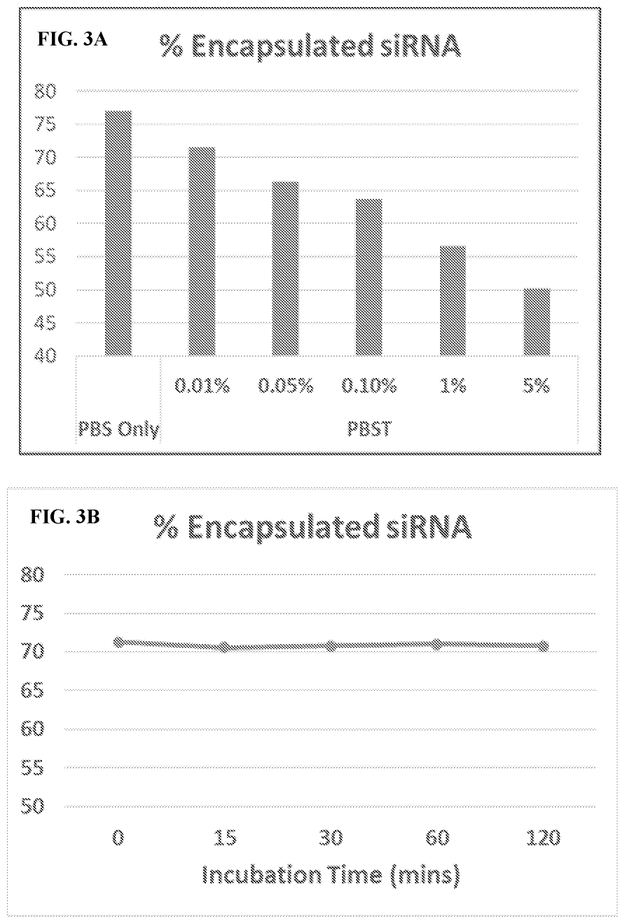

The RBP binding assay uses PBST (0.01% or 0.05%) as a wash buffer. Tween20 is a polysorbate-type nonionic surfactant, which could potentially disrupt liposome lipid bilayer therefore impacting the integrity of an LNP. An Encapsulation Efficiency (EE) test (RiboGreen Assay) was utilized to evaluate LNP integrity after exposure to PBST buffer. LNP samples were incubated in PB ST buffer with different amounts of Tween20 (0.0%, 0.01%, 0.05%, 0.1%, 1% and 5%) for 30 mins, than analyzed by RiboGreen assay. As shown in FIG. 3A, percent encapsulated RNA decreased when the concentration of Tween20 in PBST increased. Incubation in 0.01% PBS-Tween buffer (71.6%) remained comparable percent encapsulated siRNA compared with PBS buffer (76.0%), while incubation in 0.05% PB ST buffer resulted lower percent encapsulated siRNA (66.4%). This result suggests that 0.01% PBST buffer is a better wash buffer in maintaining LNP integrity compared with higher amounts of PBST buffer.

Next, an experiment was performed to evaluate if longer incubation in 0.01% PBST buffer would result in more disrupted LNP structure, hence less percent encapsulated siRNA. LNP was incubated in 0.01% PBST buffer for up to 120 mins, and no change in percent encapsulated siRNA was observed (FIG. 3B). Collectively, these data indicate that 0.01% PBST buffer had no significant impact on LNP integrity and further validates the use of 0.01% PBST as a wash buffer in the assay.

### Pre-Validation Method Optimization

Data generated during method development showed a high level of plate-to-plate variability (inter-plate CV as high as 30-45%), indicating there are factors that affect calibrator and sample testing results. Those identified factors as shown in Table 15 has been extensively evaluated and no significant improvement observed by changing most of them, except: (1) adding triton to calibration diluent. Addition of triton to calibration diluent has reduced inter-plate CV to less than 25%. However, the presence of triton has been shown to disrupt HSP47 LNP, and cause the release of siRNA. So triton was not used in the calibration diluent. Alternatively, (2) a higher concentration of capture protein and detection antibody can reduce inter-plate CV to <20%. Therefore, this was incorporated into the assay condition.

TABLE 15
Evaluation of Assay Procedures

| | | Impact on |
|---|---|---|
| | | inter-plate |
| Assay Procedure | Conditions | CV (Y/N) |
| LNP preparation | Prepare calibration curve differently | N |
| and incubation | Prepare positive control differently | N |
| | Incubate samples for different time | N |
| | or at different temperature | |
| LNP storage in water | LNP storage in water | N |
| Alternative | Pre-incubation of reagents | N |
| assay workflow | Off-line incubation of | N |
| | biotin-RBP and LNP | |
| Wash buffer | Default vs. angles vs. angled/gentle | N |
| dispense methods | | |
| Pipettes | Positive displacement pipette | N |
| Addition of | Addition of Hydroxypropyl- | N |
| Hydroxypropyl- | beta-cylodextrin in | |
| beta-cylodextrin in | calibration diluent | |
| calibration diluent | | |
| Additional | Addition of Triton | Y |
| calibrator diluent | Addition of Brij | N |
| Higher concentration | 1.5 μg/mL instead of 0.5 μg/mL | Y |
| of capture protein and | | |
| detection antibody | | |

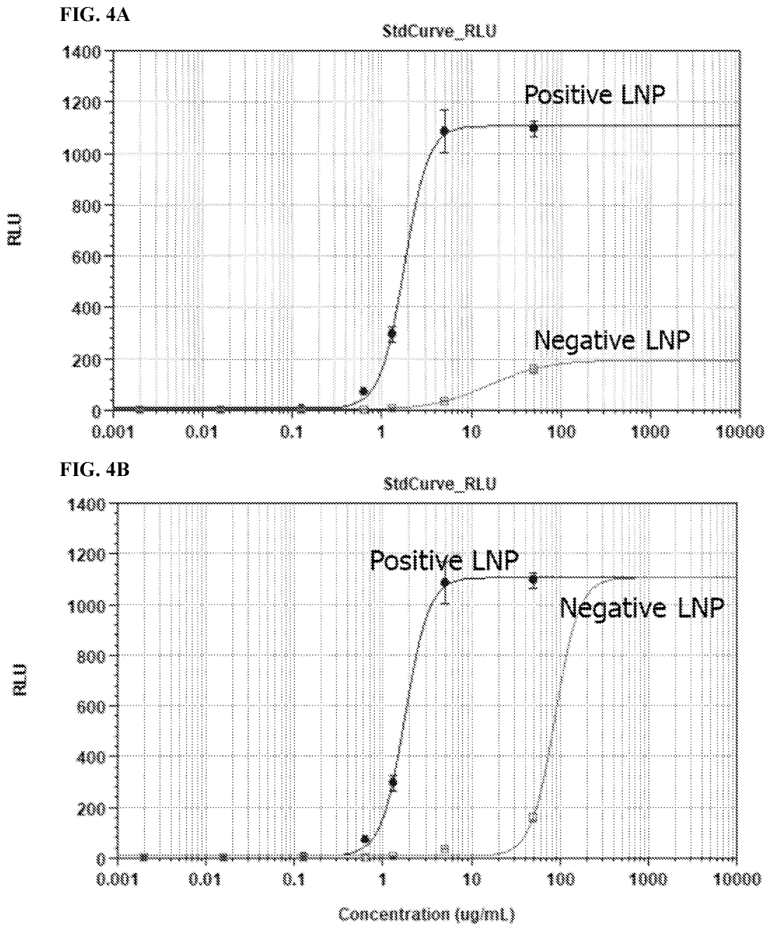

To reduce plate-to-plate variability, the assay format was modified to measure the RBP binding curve of each sample, and calculate the relative % RBP-binding by comparing the normalized EC50 value to the reference standard (positive LNP (FIGS. 4A-4B). In this assay format, the positive to negative ratio increased from 10-20 fold to >30 fold, and inter-plate CV reduced to <10%, as shown below.

### Assay Validation

The definition of relative potency (RP) requires that two samples are similar, meaning that they have parallel dose-response curves. Testing for parallelism is a prerequisite to calculate the relative potency of a compound and plays an important role in drug comparison, analyte confirmation, cross-reactivity, interfering substances, matrix compensation and concentration estimation. The relative potency is generally set to one for the reference curve (known agent) and the scaling factor used to transform the reference curve into the test curve (unknown agent) is the relative potency of the unknown agent. Noise and weighting are built into the analysis. An optimal weighting factor will ensure that the results are not dominated by the most variable data points.

In this method a parallel line analysis template was built to measure parallelism between samples in an assay. Relative is determined in the linear region of the curve where the response changes relative to the concentration at 50% effective dose or EC. The curves tested are then fitted to the constrained model. The parameters describing the curves are identical for all curves except for the X-axis in the 4-parameter curve fit equation.

All data generated during this validation were analyzed using the parallel line analysis template, and utilized to measure target bio-performance of LNP to RBP in the method, as shown in Table 16.

TABLE 16
Parallelism Analysis
F-Test

| | D Parameter | | | | |
|---|---|---|---|---|---|
| | (Upper | | B Parameter | | |
| | Asymptote) | | (Slope Factor) | | |
| | Confi- | | Confi- | | |
| Lot | dence | DRef/ | dence | BRef/ | |
| Number | Interval | DTest | Interval | BTest | Result |
| Lot 1: Vial 1 | 0.804-0.878 | 0.84 | 0.736-1.123 | 0.909 | Two curves |
| vs. Vial 2 | | | | | are parallel |
| Lot 1: Vial 1 | 1.018-1.090 | 1.053 | 0.886-1.185 | 1.024 | Two curves |
| vs. Vial 3 | | | | | are parallel |
| Lot 2: Vial 1 | 0.941-1.037 | 0.988 | 0.774-1.198 | 0.964 | Two curves |
| vs. Vial 2 | | | | | are parallel |
| Lot 1 vs. | 1.017-1.108 | 1.062 | 0.831-1.221 | 1.006 | Two curves |
| Lot 2 | | | | | are parallel |

Accuracy and precision are two important factors to consider when taking data measurements. Both accuracy and precision reflect how close a measurement is to an actual value, but accuracy reflects how close a measurement is to a known or accepted value, while precision reflects how reproducible measurements are, even if they are far from the accepted value. The objective of this study was to evaluate the precision and accuracy of the assay by measuring the relative % RBP-binding of two clinical lots (3 lots from lot 1 and 2 vials from lot 2) across 9 runs.

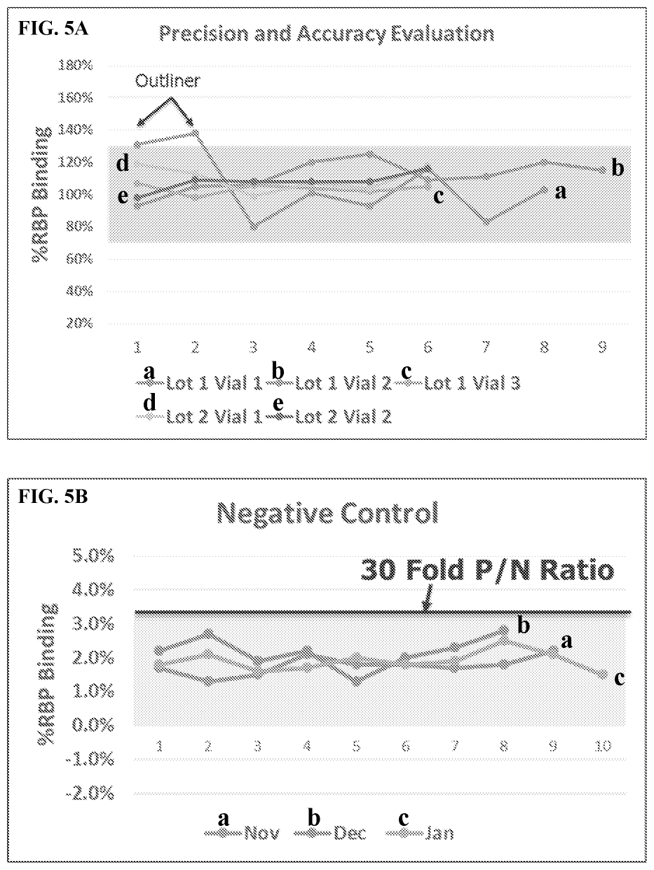

Clinical lot 1 showed higher relative % RBP-binding in two experiments compared to other experiments or other vials or clinical lots (FIG. 5A). This has been identified as sample dilution deviation and not considered in the final precision/accuracy evaluation. The precision of this assay is 9.3% and accuracy is 70% to 130%.

The negative control has also been tested in all the experiments to ensure the high specificity of RBP binding. The negative control consistently showed low RBP-binding affinity (<3%), indicating a positive/negative ratio >30 fold (FIG. 5B).

### Linearity

Linearity is an indicator of the consistency of measurements over the entire range of measurements. Linearity indicates how well the instrument measurement corresponds to reality. The objective of this study was to evaluate assay range shat shows linearity in concentration measurement at different dilutions.

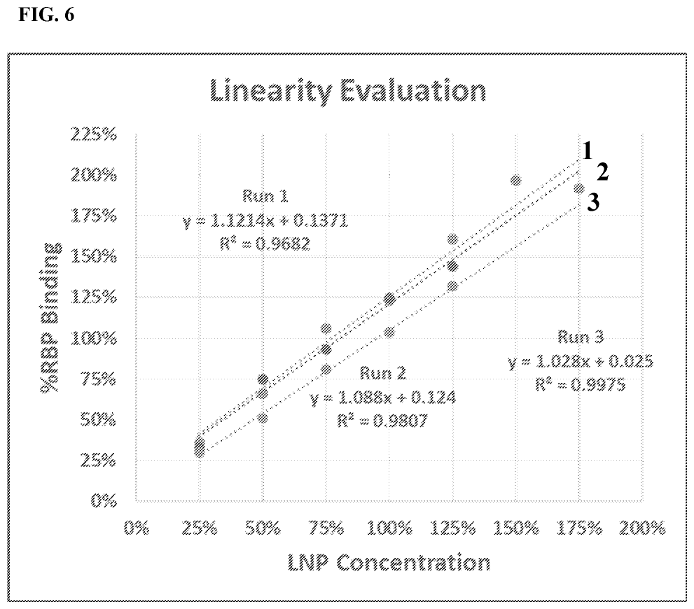

Seven samples with starting concentration of 87.5, 75, 62.5, 50, 37.5, 25 and 12.5 ug/mL were prepared from reference standard. These concentrations correspond to 25% to 175% of the recommended reference start concentration (50 ug/mL). Linearity was measured as the slope of the regression line between the relative concentrations and their measured relative % RBP-binding. The relative % RBP-binding of HSP-LNP is correlated with sample concentration, as the solution with more LNP binds more RBP (FIG. 6).

### Limit of Quantitation

The quantitation limit of an individual analytical procedure is the lowest and highest amount of analyte in a sample which can be quantitatively determined with suitable precision and accuracy. The final LLOQ is 0.181 ug/mL and ULOQ is 4.71 ug/mL.

### Assay Robustness

Robustness is the evaluation of an analytical method wherein the results obtained are found to be reliable even when performed in a slightly varied condition. It is the ability of a method to remain unaffected when slight variations are applied.

Assay robustness has been evaluated at different analytical procedure deviation as shown in Table 20. There is no impact on the relative % RBP binding after HSP LNP has been stored at room temperature for up to two days, or exposed to white light for up to 60 mins, or diluted to working concentration then stored at room temperature for up to 45 mins. The results showed high robustness of assay procedures.

TABLE 17
Robustness

| Room Temp Storage | Control | 1 day | 2 day |
|---|---|---|---|
| % RBP Binding | 100% | 98% | 114% |
| White Light Exposure | Control | 30 mins | 60 mins |
| % RBP Binding | 100% | 97% | 118% |

| Stability of Working | | | | |
|---|---|---|---|---|
| Conc. at Room Temp | Control | 15 mins | 30 mins | 45 mins |
| % RBP Binding | 100% | 107% | 91% | 91% |

### Sample Testing Using New Method

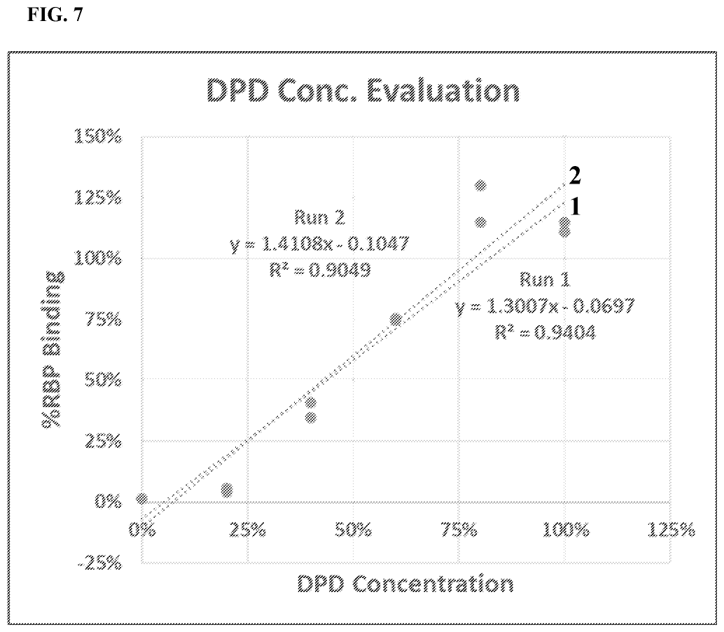

The validated ligand binding assay is to evaluate the RBP binding affinity of HSP LNP through DPD-RBP binding. An important question is to address if the amount of DPD per LNP has any impact on LNP RBP binding. HSP LNP samples were manufactured with different target % DPD (0%, 20%, 40%, 60%, and 80%), and tested by the new RBP binding assay. The relative % RBP-binding of HSP47 LNP is correlated with level of DPD on LNP, as LNP with higher % DPD showed stronger RBP binding (FIG. 7).

The DPD component of the LNP is sensitive to UV and white light (indoor light), which negatively impact the targeting ability of the DPD. Isomerization and chain oxidation reaction are the main degradation pathways induced by UV/white light exposure. In order to evaluate the impact of DPD photo-degradation on DPD-RBP binding, the photo-degraded HSP LNP sample was tested by the new RBP binding assay. As shown in Table 18, the relative % RBP-binding of HSP LNP were comparable to the control.

TABLE 18
Relative RBP Binding (%) of Photo-Degraded LNP
Sample (50% Degraded DPD) by New Method

| Sample Name | Relative RBP Binding (%) |
|---|---|
| Control | 100 |
| 50% Photo-Degraded LNP | 108 |
| Sample—Run 1 | |
| 50% Photo-Degraded LNP | 103 |
| Sample—Run 2 | |

HSP47 LNP is Lyo product, and reconstituted by adding water and gentling pipetting up and down. Harsh reconstitution procedures (Table 19) have been evaluated before to assess LNP integrity, and EE assay has confirmed no siRNA leakage. Thus LNPs remained intact under the evaluated conditions. The same procedures were further evaluated by RBP binding assay to assay if the reconstitution procedure has any impact on the drug product target bio-performance. As shown in Table 19, the samples that went through all the harsh reconstitution procedures remained comparable % RBP binding relative to control. The different harsh reconstitution procedures have no significant impact on the RBP binding affinity even after storage at room temperature for up to 16 hours.

TABLE 19
Relative RBP Binding (%) Using
Different Reconstitution Procedures

| | | 1 hour stored | 16 hours stored |
|---|---|---|---|
| Reconstitution | | at Room | at Room |
| Procedure | Initial | Temperature | Temperature |
| Control | 97 | 105 | 97 |
| Vortex for 1 minute | 86 | 102 | 99 |
| Vigorously shake back and | 96 | 104 | 103 |
| forth for 60 times | | | |
| Sonicate for 10 minutes | 98 | 100 | 94 |

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

All publications, patents, and patent applications mentioned in this specification are herein incorporated by reference to the same extent as if each individual publication, patent, or patent application was specifically and individually indicated to be incorporated by reference.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for detecting a liposome in a sample, comprising contacting the sample with a modified retinol binding protein (RBP) associated with an immobilized surface; wherein an exterior surface of the liposome comprises a retinoid or a fat-soluble vitamin; and wherein the modified RBP binds the retinoid or the fat-soluble vitamin.

2. A method of measuring the relative RBP-binding affinity of a liposome in a sample, comprising contacting the sample with a modified RBP associated with an immobilized surface; wherein an exterior surface of the liposome comprises a retinoid or a fat-soluble vitamin; and wherein the modified RBP binds the retinoid or the fat-soluble vitamin.

3. The method of claim 1 or 2, wherein the retinoid or the fat-soluble vitamin comprises a vitamin A or a DiVA-PEG-DiVA (DPD) construct.

4. The method of any one of claims 1 to 3, wherein the modified RBP comprises a heterologous moiety, and wherein the immobilized surface comprises a binding element that is capable of binding the heterologous moiety.

5. The method of claim 4, wherein the heterologous moiety comprises biotin.

6. The method of claim 4 or 5, wherein the binding element comprises avidin, streptavidin, NeutrAvidin, CaptAvidin, or any combination thereof.

7. The method of any one of claims 1 to 6, wherein the modified RBP comprises a biotin, and wherein the immobilized surface comprises avidin, streptavidin, NeutrAvidin, or any combination thereof.

8. The method of any one of claims 1 to 7, wherein the immobilized surface is a plate.

9. The method of claim 8, wherein the plate comprises one or more electrodes associated with the bottom surface of the plate.

10. The method of any one of claims 1 to 7, wherein the immobilized surface is a magnetic bead or a chip.

11. The method of any one of claims 1 to 10, wherein the liposome is a lipid nanoparticle (LNP).

12. The method of any one of claims 1 to 11, wherein the liposome does not comprise a biotin.

13. The method of any one of claims 1 to 12, further comprising contacting the liposome with an antibody that binds an antigen present on the liposome.

14. The method of claim 13, wherein the antibody specifically binds PEG (“an anti-PEG antibody”).

15. The method of claim 14, wherein the anti-PEG antibody specifically binds a methoxy group of PEG.

16. The method of claim 14 or 15, wherein the anti-PEG antibody is conjugated to a detectable tag.

17. The method of any one of claims 1 to 16, wherein the liposome comprises a biologically active molecule.

18. The method of claim 17, wherein the biologically active molecule is a chemical compound, a nucleic acid, a peptide, an amino acid, or any combination thereof.

19. The method of claim 17 or 18, wherein the biologically active molecule is DNA, RNA, or any combination thereof.

20. The method of any one of claims 17 to 19, wherein the biologically active molecule is an antisense oligonucleotide, siRNA, shRNA, miRNA, mRNA, a DNA plasmid, Cas9 nuclease, a TALEN nuclease, a zinc finger nuclease, or any combination thereof.

21. The method of any one of claims 17 to 20, wherein the biologically active molecule inhibits heat shock protein 47 (HSP47).

22. The method of claim 20, wherein the biologically active molecule is an siRNA.

23. The method of claim 22, wherein the siRNA comprises the nucleotide sequence set forth in SEQ ID NO: 1.

24. The method of any one of claims 1 to 23, comprising treating the immobilized surface with a blocker solution prior to the contacting with the sample.

25. The method of claim 24, wherein the blocker solution comprises a polysorbate

26. The method of claim 24 or 25, wherein the blocker solution comprises about 0.001% to about 0.05% TWEEN-20.

27. The method of any one of claims 24 to 26, wherein the blocker solution comprises at least about 0.01% TWEEN-20.

28. The method of any one of claims 24 to 27, wherein the blocker solution comprises about 0.05% TWEEN-20.

29. The method of any one of claims 24 to 28, further comprising washing the immobilized surface with a wash buffer prior to the contacting with the sample.

30. The method of claim 29, comprising washing the immobilized surface at least one time, at least two times, at least three times, at least four times, or at least five times with the wash buffer.

31. The method of claim 29 or 30, comprising washing the immobilized surface three times with the wash buffer.

32. The method of any one of claims 29 to 31, wherein the wash buffer comprises phosphate buffered saline (PBS) and TWEEN-20.

33. The method of any one of claims 29 to 32, wherein the wash buffer comprises about 0.001% to about 0.05% TWEEN-20.

34. The method of any one of claims 29 to 33, wherein the wash solution comprises at least about 0.01% TWEEN-20.

35. The method of any one of claims 29 to 33, wherein the wash solution comprises about 0.05% TWEEN-20.

36. The method of any one of claims 1 to 35, further comprising washing the immobilized surface with a wash buffer after the contacting with the sample.

37. The method of claim 36, comprising washing the immobilized surface at least one time, at least two times, at least three times, at least four times, or at least five times with the wash buffer.

38. The method of claim 36 or 37, comprising washing the immobilized surface three times with the wash buffer.

39. The method of any one of claims 36 to 38, wherein the wash buffer comprises PBS and TWEEN-20.

40. The method of any one of claims 36 to 39, wherein the wash buffer comprises about 0.001% to about 0.05% TWEEN-20.

41. The method of any one of claims 36 to 40, wherein the wash buffer comprises at least about 0.01% TWEEN-20.

42. The method of any one of claims 36 to 40, wherein the wash buffer comprises about 0.05% TWEEN-20.

43. The method of any one of claims 1 to 42, further comprising contacting the immobilized surface with a detection antibody solution.

44. The method of claim 43, wherein the detection antibody solution comprises an antibody that specifically binds an antigen present on a lipid-based particle.

45. The method of claim 44, wherein the antibody specifically binds PEG (“an anti-PEG antibody”).

46. The method of claim 45, wherein the anti-PEG antibody specifically binds a methoxy group of PEG.

47. The method of any one of claims 44 to 46, wherein the antibody is conjugated to a detectable tag.

48. The method of any one of claims 43 to 49, further comprising washing the immobilized surface with a wash buffer.

49. The method of claim 48, comprising washing the immobilized surface at least one time, at least two times, at least three times, at least four times, or at least five times with the wash buffer.

50. The method of claim 48 or 49, comprising washing the immobilized surface three times with the wash buffer.

51. The method of any one of claims 48 to 50, wherein the wash buffer comprises PBS and TWEEN-20.

52. The method of any one of claims 48 to 51, wherein the wash buffer comprises about 0.001% to about 0.05% TWEEN-20.

53. The method of any one of claims 48 to 52, wherein the wash buffer comprises at least about 0.01% TWEEN-20.

54. The method of any one of claims 48 to 53, wherein the wash buffer comprises about 0.05% TWEEN-20.

55. The method of any one of claims 44 to 54, further comprising detecting the antibody.

56. The method of any one of claims 1 to 55, further comprising administering the liposome to a subject in need thereof.

57. The method of any one of claims 1 to 56, wherein the liposome is capable of preventing or treating a disease or condition in a subject.

58. The method of claim 57, wherein the disease or condition affects the liver of the subject.

59. The method of claim 57 or 58, wherein the disease or condition is selected from hepatic cirrhosis, liver fibrosis, Nonalcoholic Steatohepatitis (NASH), alcoholic steatohepatitis, primary sclerosing cholangitis, primary biliary cirrhosis, and any combination thereof.

60. The method of claim 57, wherein the disease or condition comprises an organ fibrosis.

61. The method of claim 60, wherein the organ fibrosis is selected from liver fibrosis, lung fibrosis, a glial scar, arterial stiffness, arthrofibrosis, Crohn's disease, Dupuytren's contracture, keloid formation, mediastinal fibrosis, myelofibrosis, peyronie's disease, nephrogenic systemic fibrosis, progressive massive fibrosis, retroperitoneal fibrosis, scleroderma/systemic sclerosis, adhesive capsulitis, and any combination thereof.

62. A kit for measuring the relative RBP-binding affinity of a liposome in a sample according to the method of any one of claims 1 to 61.

63. A kit for assaying the relative RBP-targeting bioperformance of a liposome in a sample according to the method of any one of claims 1 to 61.
